(19) United States
(12) Patent Application Publication
(10) Pub. No.: US 2006/0193844 A1
(43) Pub. Date: Aug. 31, 2006

(54) Methods for enzyme inhibition

(75) Inventors: Mark K. Bennett, Moraga, CA (US); Christopher J. Kirk, Millbrae, CA (US); Susan D. Demo, San Francisco, CA (US)

Correspondence Address: FISH & NEAVE IP GROUP;ROPES & GRAY LLP, ONE INTERNATIONAL PLACE, BOSTON, MA, 02110-2624, US

(73) Assignee: Proteolix, Inc., South San Francisco, CA (US)

(21) Appl. No.: 11/365,789

(22) Filed: Feb. 28, 2006

Related U.S. Application Data
(60) Provisional application No. 60/657,110, filed on Feb. 28, 2005.

(51) Int. Cl. A61K 38/54

(52) U.S. Cl. 424/94.2; 514/2

(57) ABSTRACT

Improved regimens for administering proteasome inhibitors are described, wherein proteasome inhibition is more sustained relative to certain current regimens which permit substantial recovery of proteasome activity between doses of inhibitor.

### CROSS REFERENCE TO RELATED APPLICATION

This application claims the benefit of U.S. Provisional Application No. 60/657,110, filed Feb. 28, 2005, the specification of which is hereby incorporated by reference in its entirety.

### BACKGROUND OF THE INVENTION

In recent years, the proteasome has become an appealing target for therapeutic intervention in cancer, immune and auto-immune disorders, inflammation, ischemic conditions, neurodegenerative disorders and other diseases. To date, however, the only FDA-approved proteasome inhibitor is bortezomib (VELCADE™) for the treatment of relapsed multiple myeloma patients. Thus, much of what is known about therapeutic proteasome inhibition is based on work with this molecule.

Bortezomib is a boronic acid derivative that reversibly inhibits the 26S proteasome. The primary target is the chymotryptic-like activity of the 20S core peptidases. This enzymatic inhibition affects multiple signaling pathways, ultimately resulting in cell death. The maximum tolerated dose (MTD) is 1.3 mg/m²/dose administered twice weekly for two weeks (days 1, 4, 8 and 11) followed by a ten-day rest period. This twice-weekly clinical dosing regimen was recommended in order to allow the proteasome activity to recover between dose administrations, resulting in improved tolerability (Velcade SBA Medical Review). Indeed, daily administration of doses that result in 80% inhibition of the proteasome result in 67% lethality in female rats (SBA, Pharmacology Review, Part 1, p 21) and the highest dose without severe toxicity for daily administered bortezomib in monkeys is 0.5 mg/m², a dose 20-fold less than the MTD on the clinical dosing schedule (SBS Pharmacology Review, Part 3, p 111).

Frequency and severity of toxicities appear to be dose-related. Thus, it has been presumed that these toxicities may be related to the extent of proteasome inhibition (Orlowski, et.al., JCO (2002) 20: 4420-7.) and (U.S. Pat. No. 6,613,541 B1, Vaddi et al, Sep. 2, 2003). At the recommended dose and schedule, the percent of proteasome inhibition averages 80% and fully recovers between doses.

An improved dosing regimen that reduces toxicity or permits the administration of higher doses of inhibitor would be useful in improving the success of therapies that rely on proteasome inhibition.

### SUMMARY OF THE INVENTION

The invention generally relates to methods for administering proteasome inhibitors in a manner that prevents full recovery of proteasome activity between two or more successive doses of an inhibitor. As used herein, proteasome activity is preferably the chymotryptic activity of the 26S proteasome, and a proteasome inhibitor is any compound that inhibits 26S proteasome activity, preferably by binding to the 26S proteasome. In certain embodiments, the invention contemplates a series of administrations during which full recovery of activity is not permitted, while in other embodiments, full recovery of activity is permitted between some successive doses, but not between others. In general, it is contemplated that successive administrations within about 72 hours, about 48 hours, or preferably within about 24 or even about 12 hours prevent full recovery of proteasome activity between doses.

In one aspect, the invention provides methods that involve administering to or contacting a subject, a cell, a tissue, an organ or an organism with an effective amount of a composition comprising one or more proteasome inhibitors. These methods include, but are not limited to, inhibiting or reducing HIV infection in a subject; affecting the level of viral gene expression in a subject; altering the variety of antigenic peptides produced by the proteasome in an organism; treating a neurodegenerative disease in a subject; reducing the rate of intracellular protein degradation in a cell; reducing the rate of p53 protein degradation in a cell; suppressing the immune system of a subject (e.g., conditions such as septic shock, psoriasis, graft rejection, and rheumatoid arthritis); inhibiting IκB-α degradation in an organism and reducing the content of NF-κB in a cell, muscle, organ or subject; treating proliferative disease in a subject; affecting proteasome-dependent regulation of oncoproteins in a cell; treating cancer growth in a subject; and treating p53-related apoptosis in a subject.

Other features and advantages of the invention will be apparent from the following detailed description and from the claims.

### BRIEF DESCRIPTION OF THE DRAWINGS

FIG. 1 exemplifies two possible dosing regimens according to the invention and schematically depicts their effects on proteasome activity.

FIG. 2 shows effects of proteasome inhibitor exposure time as related to cell viability.

FIG. 3 depicts the effects of various dosing regimens using different proteasome inhibitors in vitro.

FIG. 4 illustrates the effects of various dosing regimens using different proteasome inhibitors in an in vivo tumor model.

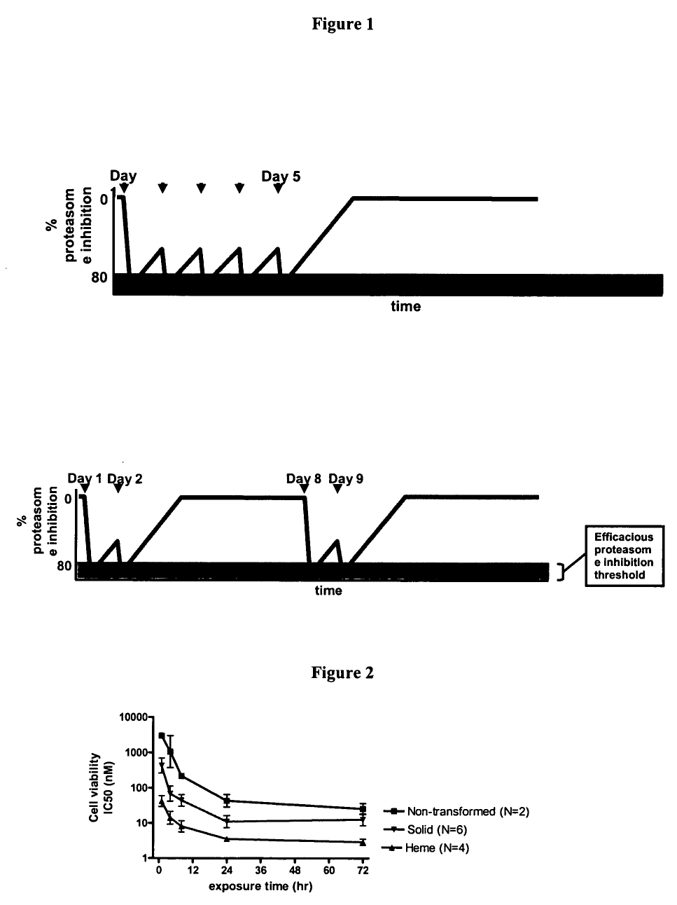

### DETAILED DESCRIPTION

The present invention is based on the finding that, despite prior assumptions, increasing the length of time of proteasome inhibition can be tolerated in animal models.

In contrast to the experience with bortezomib that proteasome inhibition cannot exceed 80% (NDA No. 21-602) and must be followed by complete recovery before redosing in order to avoid unacceptable toxicity, it has been found that preventing full proteasome recovery between doses is both tolerated and more efficacious in these models.

There are two ways that proteasome activity can be restored once a cell or organism has been exposed to an inhibitor. In the case of reversibly-binding inhibitors, proteasome activity can be immediately restored by equilibration once the inhibitor is removed. However, for inhibitors that bind irreversibly, restoration of proteasome activity cannot occur via this mechanism.

Another means by which proteasome activity can be restored is by the de novo synthesis/assembly of new proteasomes. In experiments where proteasome activity has been nearly completely inhibited by use of an irreversibly-binding proteasome inhibitor, the t½ (the time at which 50% of normal proteasome activity is restored) is estimated to be approximately 24 hours based on the recovery of proteasome activity.

Accordingly, one embodiment of the invention is a method of inhibiting proteasome activity by administering an initial dose of a proteasome inhibitor followed by a subsequent administration of a proteasome inhibitor within about 72 hours of the initial dose, preferably within about 48 hours of the initial dose, and most preferably within about 24 hours of the initial dose. There may be further subsequent administrations as well, each preferably within about 72 hours of the previous dose, preferably within about 48 hours of the previous dose, and most preferably within about 24 hours of the previous dose.

The term “about” as used herein is meant to be plus or minus 6 hours, 4 hours, 1 hour, or even 30 minutes, such that, for example, the phrase “about 24 hours” includes the time period of 18 to 30 hours.

Subsequent administrations of a proteasome inhibitor need not be at the same dose level as the initial dose, nor be administered via the same route of delivery as the initial dose. Additionally, subsequent administrations may be of a proteasome inhibitor that is a different compound or of a different class of compound from the previously administered proteasome inhibitor. Indeed, since side effects not directly related to proteasome inhibition would be expected to vary between different inhibitors, administering a variety of proteasome inhibitors may be useful to limit unwanted side effects.

Proteasome activity can be measured by any of a number of assays well known in the art. U.S. Pat. No. 6,613,541 discloses such an assay, which can be conveniently performed on a biological sample such as a blood sample. In this way, a patient's proteasome activity can be monitored so as to administer a proteasome inhibitor before proteasome activity has returned to an uninhibited level, or to ensure that the dosage is sufficient to achieve the desired level of suppression.

Proteasome Inhibitors

Suitable proteasome inhibitors, particularly those with epoxide and aziridine moieties, are described in WO 05/111,088, filed May 9, 2005, U.S. Pat. No. 6,831,099, and U.S. patent application No., 11/106,879, filed Apr. 14, 2005, Ser. No. 11/199,899, filed Aug. 8, 2005, and Ser. No. 11/254,541, filed Oct. 19 2005, the contents of which are incorporated herein by reference.

In each of the following groups, the values for various moieties (e.g., for R¹, etc.) are understood to be consistent within a group, but values for one group (e.g., Group 1) do not apply to another group (Group 9).

Group 1

In one embodiment, the proteasome inhibitor is an epoxide- or aziridine-containing compound, which preferably contains groups proximate to the heteroatom-containing, three-membered rings, such that a ring-opening reaction of the heteroatom-containing three-membered ring is facilitated. Such groups include electron-withdrawing groups (E.W.G.) adjacent to (for example, at a carbon vicinal to a carbon atom of the three-membered, heteroatom-containing ring), or in electronic communication with (for example, via a carbon atom, or an alkenyl or alkynyl linkage), epoxide or aziridine functionalities. The E.W.G. can be bonded to one of the carbon atoms of the heteroatom-containing, three-membered ring. E.W.G. include, for example, cyano, isocyano, nitro, amide, sulfonyl, β-carboxy vinyl, sulfinyl, β,β-dicyano vinyl, formyl, carboxyl, alkyloxy- and aryloxy-carbonyl, 1-tetrazolyl, carbamoyl, sulfamoyl, carbonyl, sulfoxide groups, and halogenated or dihalogenated carbon atoms such as —CHX—, —CXX′—, and —CRX— (where X and X′ are independently selected halogens, and R is a carbon-containing substituent such as alkyl, aryl alkenyl, alkynyl and the like). In some preferred embodiments, E.W.G. is a carbonyl group.

In some embodiments, it may be desirable to utilize E.W.G. that are of size, charge, and polarity sufficient to interact electronically with particular polar or charged moieties within an Ntn hydrolase. For example, an ionized aspartate or glutamate side chain can be present in the Ntn, and interact with, and stabilize, an electron-withdrawing group present in a peptide epoxide. Such groups act as an “anion hole,” with which E.W.G. can interact when enzyme inhibitors are bound to Ntn, resulting in increased electrophilicity of E.W.G.

Some peptide epoxide or peptide aziridine compounds have a ketone functionality as the electron-withdrawing group, along with epoxide or aziridine functional groups. Particular examples are peptide α′,β′-epoxy ketones or peptide α′,β′-aziridine ketones, in which the carbon atoms forming two of the three members of the epoxide or aziridine ring are one (α′) and two (β′) carbons from the ketone, and the ketone carbon is bonded to one of the carbon atoms of the heteroatom-containing, three-membered ring. Further groups can be bonded to α′ or β′ carbons such as hydrogen and branched or unbranched C₁₋₄ alkyl groups, including methyl, ethyl, propyl and butyl groups. Groups bonded to α′ or β′ carbons can be further substituted with hydroxy, halogen, amino, carboxy, carbonyl, thio, sulfide, ester, amide or ether functionality.

For example, a carboxylic acid group can be bonded directly to the α′ carbon, or via a linker. The linker can be C₁₋₄ alkylene, C₂₋₅ alkenylene, C₂₋₅ alkynylene, aryl, oxygen, sulfur, or amine. This carboxylic acid can be part of a peptide moiety extending from the α′ carbon of the heteroatom-containing, three-membered ring. In this way, peptides containing side chains can be constructed. Such side chains can be labeled as P1′, P2′, and so forth, with P1′ being the first side chain adjacent to the α′ carbon, P2′ being the second, and so forth. Optimization of side chains for P1′, P2′ and other positions can result in enzyme inhibitors with desirable specificity, or desirable inhibition rates. Side chains for P1′, P2′ and so forth can be any of those side chains discussed herein.

In embodiments including such groups bonded to α′ carbons, the stereochemistry of the α′-carbon (that carbon forming a part of the epoxide or aziridine ring) can be (R) or (S). Note that a preferred compound may have a number of stereocenters having the indicated up-down (or β-α, where β as drawn herein is above the plane of the page) or (R)—(S) relationship (that is, it is not required that every stereocenter in the compound conform to the preferences stated). In some preferred embodiments, the stereochemistry of the α′ carbon is (R), that is, the X atom is β, or above the plane of the molecule, when drawn as below. For example, the following general structural formula (I) demonstrates a preferred stereochemistry for some embodiments: 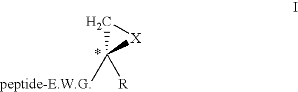
where X is oxygen or an NH or N-alkyl group, E.W.G. is an electron withdrawing group as described above, “peptide” is a peptide as describe below, and R is a hydrogen atom, a branched or unbranched C₁₋₄ alkyl group, which can be further substituted with hydroxy, halogen, amino, carboxy, carbonyl, thio, sulfide, ester, amide or ether functionality. For some embodiments, the X atom should be configured as above in order to facilitate interaction with an N-terminal nucleophilic group in an Ntn hydrolase. For example, irreversible interactions of enzyme inhibitors with the β5/Pre2 subunit of 20S proteasome which lead to inhibition appear to be facilitated by the configuration illustrated above. In the case of other Ntn hydrolases, the opposite stereochemistry of the α-carbon of the peptide epoxides or peptide aziridines may be preferred.

In the case illustrated above, the β′ carbon is substituted with two hydrogen atoms. Regarding the stereochemistry, the chiral α′ carbon is indicated with a star, and the Cahn-Ingold-Prelog rules for determining absolute stereochemistry are followed. These rules are described, for example, in Organic Chemistry, Fox and Whitesell; Jones and Bartlett Publishers, Boston, Mass. (1994); Section 5-6, pp 177-178, which section is hereby incorporated by reference. The stereochemistry of the α′ carbon is (R) when the oxygen or nitrogen has the highest priority, the peptide-E.W.G. group has second highest priority, and the —CH₂—X— group has third highest priority. If the relative priorities of the peptide-E.W.G., —CH₂—X—, and R groups change, the nominal stereochemistry can change, but the essential configuration of the groups can remain the same, for some preferred embodiments. That is, referring to the general structure immediately above, peptide-E.W.G. is joined to the chiral α′ carbon from the left, R is joined to the chiral α′ carbon from the right, and the X atom(s) project(s) from the plane of the page. The nitrogen atom of an aziridine ring can also, in principle, be chiral, as discussed in March, Advanced Organic Chemistry, 4^{th} Ed. (1992) Wiley-Interscience, New York, pp. 98-100, which pages are incorporated herein by reference.

The peptide epoxides or peptide aziridines also include a peptide moiety. The peptide moiety is bonded to the electron-withdrawing group. The bond is made between the electron-withdrawing group and any portion of the peptide. For example, in some preferred embodiments, the E.W.G. is bonded to the terminal backbone unit, such as for example, to the carboxy terminus of the peptide. Alternatively, the E.W.G. can be bonded to the amino terminus of the peptide. In other embodiments, the E.W.G. can be bonded to a side chain of the peptide moiety.

Peptides can have a repeating backbone structure with side chains extending from the backbone units. Generally, each backbone unit has a side chain associated with it, although in some cases, the side chain is a hydrogen atom. In other embodiments, not every backbone unit has an associated sidechain. Peptides useful in peptide epoxides or peptide aziridines have two or more backbone units. In some embodiments useful for inhibiting chymotrypsin-like (CT-L) activity of the proteasome, between four and eight backbone units are present, and in some preferred embodiments for CT-L inhibition, between four and six backbone units are present. In other embodiments useful for inhibiting the PGPH activity of the proteasome, between two and eight backbone units are present, and in some preferred embodiments for PGPH inhibition, between three and six backbone units are present.

The side chains extending from the backbone units can include natural aliphatic or aromatic amino acid side chains, such as hydrogen (glycine), methyl (alanine), iso-propyl (valine), sec-butyl (isoleucine), iso-butyl (leucine), phenylmethyl (phenylalanine), and the side chain constituting the amino acid proline. The side chains can also be other branched or unbranched aliphatic or aromatic groups such as ethyl, n-propyl, n-butyl, t-butyl, and aryl substituted derivatives such as 1-phenylethyl, 2-phenylethyl, (1-naphthyl)-methyl, (2-naphthyl)-methyl, 1-(1-naphthyl)ethyl, 1-(2-naphthyl)ethyl, 2-(1-naphthyl)ethyl, 2-(2-naphthyl)ethyl, and similar compounds. The aryl groups can be further substituted with branched or unbranched C₁₋₆ alkyl groups, or substituted alkyl groups, such as acetyl and the like, or further aryl groups, or substituted aryl groups, such as benzoyl and the like. Heteroaryl groups can also be used as side chain substituents. Heteroaryl groups include nitrogen-, oxygen-, and sulfur-containing aryl groups such as thienyl, benzothienyl, naphthothienyl, thianthrenyl, furyl, pyranyl, isobenzofuranyl, chromenyl, pyrrolyl, imidazolyl, pyrazolyl, pyridyl, pyrazinyl, indolyl, purinyl, quinolyl, and the like.

In some embodiments, polar or charged residues can be introduced into the peptide epoxides or peptide aziridines. For example, naturally occurring amino acids such as hydoxy-containing (Thr, Tyr, Ser) or sulfur-containing (Met, Cys) can be introduced, as well as non-essential amino acids, for example taurine, carnitine, citrulline, cystine, ornithine, norleucine and others. Non-naturally occurring side chain substituents with charged or polar moieties can also be included, such as, for example, C₁-C₆ alkyl chains or C₆-C₁₂ aryl groups with one or more hydroxy, short chain alkoxy, sulfide, thio, carboxyl, ester, phospho, amido or amino groups, or such substituents substituted with one or more halogen atoms. In some preferred embodiments, there is at least one aryl group present in a side chain of the peptide moiety.

In some embodiments, the backbone units are amide units [—NH—CHR—C(═O)—], in which R is the side chain. Such a designation does not exclude the naturally occurring amino acid proline, or other non-naturally occurring cyclic secondary amino acids, which will be recognized by those of skill in the art.

In other embodiments, the backbone units are N-alkylated amide units (for example, N-methyl and the like), olefinic analogs (in which one or more amide bonds are replaced by olefinic bonds), tetrazole analogs (in which a tetrazole ring imposes a cis-configuration on the backbone), or combinations of such backbone linkages. In still other embodiments, the amino acid α-carbon is modified by α-alkyl substitution, for example, aminoisobutyric acid. In some further embodiments, side chains are locally modified, for example, by Δ^{E} or Δ^{Z} dehydro modification, in which a double bond is present between the α and β atoms of the side chain, or for example by Δ^{E} or Δ^{Z} cyclopropyl modification, in which a cyclopropyl group is present between the α and β atoms of the side chain. In still further embodiments employing amino acid groups, D-amino acids can be used. Further embodiments can include side chain-to-backbone cyclization, disulfide bond formation, lactam formation, azo linkage, and other modifications discussed in “Peptides and Mimics, Design of Conformationally Constrained” by Hruby and Boteju, in “Molecular Biology and Biotechnology: A Comprehensive Desk Reference”, ed. Robert A. Meyers, VCH Publishers (1995), pp. 658-664, which is hereby incorporated by reference.

The enzyme inhibitors for inhibition of chymotrypsin-like (CT-L) activity of Ntn include at least four backbone units. In some particularly preferred CT-L inhibitor embodiments, at least four amide units and an α′,β′-epoxy ketone or α′,β′-aziridine ketone moiety are present (tetrapeptide epoxy ketones or tetrapeptide aziridine ketones). In particular CT-L inhibitor embodiments with at least four amide units, the peptide moiety, and the ketone and epoxide or aziridine functionalities of the enzyme inhibitors form compounds with structural formula (II): 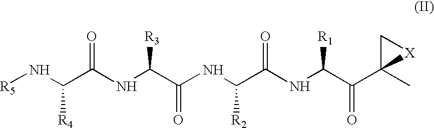
where X is oxygen, NH, or N-alkyl, R₁, R₂, R₃ and R₄ are independently selected from the group consisting of branched or unbranched C₁₋₆ alkyl or branched or unbranched C₁₋₆ hydroxy alkyl or branched or unbranched C₁₋₆alkoxy alkyl, aryl, and aryl-substituted branched or unbranched C₁₋₆alkyl, wherein such groups can further include: amide linkages; amines; carboxylic acids and salts thereof; carboxyl esters, including C₁₋₅alkyl esters and aryl esters; thiols and thioethers; and R₅ is a further chain of amino acids, hydrogen, acetyl, or a protecting group, such as N-terminal protecting groups known in the art of peptide synthesis, including t-butoxy carbonyl (BOC), benzoyl (Bz), fluoren-9-ylmethoxycarbonyl (Fmoc), triphenylmethyl(trityl) and trichloroethoxycarbonxyl (Troc) and the like. The use of various N-protecting groups, e.g., the benzyloxy carbonyl group or the t-butyloxycarbonyl group (BOC), various coupling reagents, e.g., dicyclohexylcarbodiimide, 1,3-diisopropylcarbodiimide (DIC), 1-(3-dimethylaminopropyl)-3-ethylcarbodiimide (EDC), N-hydroxyazabenzotriazole (HATU), carbonyldiimidazole, or 1-hydroxybenzotriazole monohydrate (HBT), and various cleavage reagents: for example, trifluoroacetic acid; HCL in dioxane; hydrogenation on Pd—C in organic solvents, such as methanol or ethyl acetate; boron tris(trifluoroacetate); and cyanogen bromide, and reaction in solution with isolation and purification of intermediates is well-known classical peptide methodology.

In some embodiments of chymotrypsin-like activity inhibitors, R₁ is branched or unbranched C₁₋₆alkyl. In some embodiments of chymotrypsin-like activity inhibitors, R₁ is isobutyl. In some embodiments of chymotrypsin-like activity inhibitors, R₂ is branched or unbranched C₁₋₆alkyl or aryl. In some embodiments of chymotrypsin-like activity inhibitors, R₂ is phenyl, phenylmethyl, or 1-naphthyl. In some embodiments of chymotrypsin-like activity inhibitors, R₃ is branched or unbranched C₁₋₆ alkyl or aryl. In some embodiments of chymotrypsin-like activity inhibitors, R₃ is isobutyl, phenyl or 1-naphthyl. In some embodiments of chymotrypsin-like activity inhibitors, R₄ is branched or unbranched C₁₋₆alkyl, aryl, and aryl-substituted branched or unbranched C₁₋₆alkyl. In some embodiments of chymotrypsin-like activity inhibitors, R₄ is isobutyl, phenyl, 1-naphthyl, phenylmethyl, or 2-phenylethyl. In some embodiments of chymotrypsin-like activity inhibitors, R₅ is hydrogen, C₁₋₆alkanoyl, substituted or unsubstituted aryl, substituted or unsubstituted heteroaryl, where substituents include halogen, carbonyl, nitro, hydroxy, aryl, and C₁₋₅alkyl. In some embodiments of chymotrypsin-like activity inhibitors, R₅is hydrogen, acetyl, substituted or unsubstituted aryl.

In some preferred embodiments of chymotrypsin-like activity inhibitors, simultaneously, R₁ is isobutyl, R₂ is phenylmethyl, R₃ is isobutyl, and R₄ is 2-phenylethyl, and R₅is acetyl. The peptide having such values is referred to herein as peptide (b).

In some embodiments of PGPH activity inhibitors, R₁ is hydrogen, branched or unbranched C₁₋₆alkyl. In some embodiments of PGPH activity inhibitors, R₁ is isobutyl. In some embodiments of PGPH activity inhibitors, R₂ is hydrogen, branched or unbranched C₁₋₆ alkyl or aryl. In some embodiments of PGPH activity inhibitors, R₂ is phenyl, phenylmethyl, or 1-naphthyl. In some embodiments of PGPH activity inhibitors, R₃ is hydrogen, branched or unbranched C₁₋₆cyclic alkylene bonded to the R₃ backbone unit. In some embodiments of PGPH activity inhibitors, R₃ is ethylene bonded to the amine of the R₃ amino acid backbone, such as would be the case for the amino acid proline. In some optional embodiments of PGPH activity inhibitors, R₄ is hydrogen, branched or unbranched C₁₋₆alkyl, aryl, and aryl-substituted branched or unbranched C₁₋₆alkyl. In some other optional embodiments of PGPH activity inhibitors, R₄ is hydrogen, or isopropyl. In some optional embodiments of PGPH activity inhibitors, R₅ is hydrogen, C₁₋₆alkanoyl, substituted or unsubstituted aryl, substituted or unsubstituted heteroaryl, where substituents include halogen, carbonyl, monosubstituted-, disubstituted- or unsubstituted-amino, nitro, hydroxy, aryl, and C₁₋₅ alkyl. In some optional embodiments of PGPH activity inhibitors, R₅is acetyl, N-acetyl-piperidinecarbonyl, N-dimethylaminobenzyl, isooctanoic, or benzoylbenzoic.

In some preferred embodiments of PGPH activity inhibitors, simultaneously, R₁ is isobutyl, R₂ is phenyl, R₃ is ethylene bonded to the R₃ amine of the amino acid backbone, and R₄ is hydrogen, and R₅ is acetyl.

Group 2

In another embodiment, the proteasome inhibitor used in the invention includes at least four backbone units and has a structure of formula (III) or a pharmaceutically acceptable salt thereof,

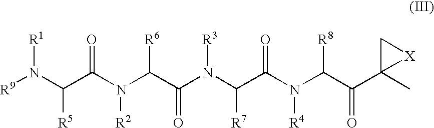

where X is O, NH, or N-alkyl, preferably O;

R¹, R², R³, and R⁴ are independently selected from hydrogen and a group of formula (IIIa), with the proviso that at least one of R¹, R², R³, and R⁴ is a group of formula (IIIa);

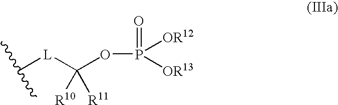

R⁵, R⁶, R⁷, and R⁸ are independently selected from branched or unbranched C₁₋₆alkyl, branched or unbranched C₁₋₆ hydroxyalkyl, branched or unbranched C₁₋₆alkoxyalkyl, aryl, and branched or unbranched C₁₋₆aralkyl, each of which is optionally substituted with a group selected from amide, amine, carboxylic acid or a pharmaceutically acceptable salt thereof, carboxyl ester, thiol, and thioether;

R⁹ is a further chain of amino acids, hydrogen, C₁₋₆acyl, a protecting group, substituted or unsubstituted aryl, or substituted or unsubstituted heteroaryl, where substituents include halogen, carbonyl, nitro, hydroxy, aryl, and branched or unbranched C₁₋₅ alkyl;

R¹⁰ and R¹¹ are independently selected from hydrogen and branched or unbranched C₁₋₆alkyl, or R¹⁰ and R¹¹ together form a 3- to 6-membered carbocyclic or heterocyclic ring;

R¹² and R¹³ are independently selected from hydrogen, a metal cation, branched or unbranched C₁₋₆alkyl, and branched or unbranched C₁₋₆aralkyl, or R¹² and R¹³ together represent C₁₋₆alkyl, thereby forming a ring; and

L is absent or is selected from —CO₂ or —C(═S)O.

Suitable N-terminal protecting groups known in the art of peptide synthesis, include t-butoxy carbonyl (Boc), benzoyl (Bz), fluoren-9-ylmethoxycarbonyl (Fmoc), triphenylmethyl (trityl) and trichloroethoxycarbonyl (Troc) and the like. The use of various N-protecting groups, e.g., the benzyloxy carbonyl group or the t-butyloxycarbonyl group (Boc), various coupling reagents, e.g., dicyclohexylcarbodiimide (DCC), 1,3-diisopropylcarbodiimide (DIC), 1-(3-dimethylaminopropyl)-3-ethylcarbodiimide (EDC), N-hydroxyazabenzotriazole (HATU), carbonyldiimidazole, or 1-hydroxybenzotriazole monohydrate (HOBT), and various cleavage conditions: for example, trifluoroacetic acid (TFA), HCl in dioxane, hydrogenation on Pd—C in organic solvents (such as methanol or ethyl acetate), boron tris(trifluoroacetate), and cyanogen bromide, and reaction in solution with isolation and purification of intermediates are well-known in the art of peptide synthesis, and are equally applicable to the preparation of the subject compounds.

In some embodiments, any two of R¹, R², R³, and R⁴ are hydrogen and any two of R¹, R², R³, and R⁴ have a structure of formula (IIIa). In preferred embodiments any three of R¹, R², R³, and R⁴ are hydrogen and any one of R¹, R², R³, and R⁴ has a structure of formula (IIIa). In the most preferred embodiment, R¹ has a structure of formula (IIIa) and R², R³, and R⁴ are hydrogen.

In certain embodiments, R⁵, R⁶, R⁷, and R⁸ are branched or unbranched C₁₋₆alkyl or branched or unbranched C₁₋₆aralkyl. In preferred embodiments, R⁶ and R⁸ are branched or unbranched C₁₋₆alkyl and R⁵ and R⁷ are branched or unbranched C₁₋₆aralkyl. In the most preferred embodiment, R⁶ and R⁸ are isobutyl, R⁵is 2-phenylethyl, and R⁷ is phenylmethyl. In certain embodiments, R⁹ is selected from hydrogen, C₁₋₆acyl, or a protecting group. In preferred embodiments, R⁹ is hydrogen or acetyl. In the most preferred embodiment, R⁹ is acetyl.

In certain embodiments, R¹⁰ and R¹¹ are selected from hydrogen and branched or unbranched C₁₋₆alkyl. In a preferred embodiment, R¹⁰ is hydrogen and R¹¹ is branched or unbranched C₁₋₆alkyl. In a further preferred embodiment, R¹⁰ is hydrogen and R¹¹ is methyl. In another preferred embodiment, both R¹⁰ and R¹¹ are hydrogen. In certain embodiments, R¹² and R¹³ are branched or unbranched C₁₋₆alkyl, metal cation, or branched or unbranched C₁₋₆aralkyl. In certain preferred embodiments, R¹² and R¹³ are selected from benzyl, tert-butyl, and sodium cation. In more preferred embodiments, both R¹² and R¹³ are benzyl or tert-butyl. In the most preferred embodiment, at least one of R¹² and R¹³ is a sodium cation.

In certain embodiments, a compound of formula (III) has the following stereochemistry:

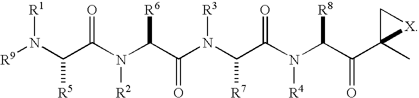

In preferred embodiments, the inhibitor has a structure of formula (IV) or a pharmaceutically acceptable salt thereof,

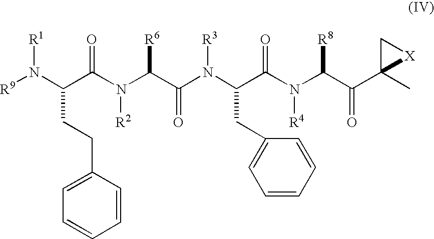

where X is O, NH, or N-alkyl, preferably O;

R¹, R², R³, and R⁴ are independently selected from hydrogen and a group of formula (IIIa), with the proviso that at least one of R¹, R², R³, and R⁴ is a group of formula (IIIa);

R⁶ and R⁸ are independently selected from branched or unbranched C₁₋₆alkyl, branched or unbranched C₁₋₆hydroxy alkyl, branched or unbranched C₁₋₆alkoxyalkyl, aryl, and branched or unbranched C₁₋₆aralkyl, each of which is optionally substituted with a group selected from amide, amine, carboxylic acid or a pharmaceutically acceptable salt thereof, carboxyl ester, thiol, and thioether;

R⁹ is a further chain of amino acids, hydrogen, acyl, a protecting group, substituted or unsubstituted aryl, or substituted or unsubstituted heteroaryl, where substituents include halogen, carbonyl, nitro, hydroxy, aryl, and branched or unbranched C₁₋₅ alkyl. Suitable N-terminal protecting groups known in the art of peptide synthesis, include t-butoxy carbonyl (Boc), benzoyl (Bz), fluoren-9-ylmethoxycarbonyl (Fmoc), triphenylmethyl (trityl) and trichloroethoxycarbonyl (Troc) and the like; and

In some embodiments of chymotrypsin-like activity inhibitor prodrugs, any two of R¹, R², R³, and R⁴ are hydrogen and any two of R¹, R², R³, and R⁴ have a structure of formula (IIIa). In preferred embodiments any three of R¹, R², R³, and R⁴ are hydrogen and any one of R¹, R², R³, and R⁴ has a structure of formula (IIIa). In the most preferred embodiment, R¹ has a structure of formula (IIIa) and R², R³, and R⁴ are hydrogen.

In certain embodiments, R⁶ and R⁸ are branched or unbranched C₁₋₆alkyl or branched or unbranched C₁₋₆araalkyl. In preferred embodiments, R⁶ and R⁸ are branched or unbranched C₁₋₆alkyl. In the most preferred embodiment, R⁶ and R⁸ are isobutyl. In certain embodiments, R⁹ is selected from hydrogen, C₁₋₆acyl, or a protecting group. In preferred embodiments, R⁹ is hydrogen or acetyl. In the most preferred embodiment, R⁹ is acetyl.

In certain embodiments, R¹⁰ and R¹¹ are selected from hydrogen and branched or unbranched C₁₋₆alkyl. In a preferred embodiment, R¹⁰ is hydrogen and R¹¹ is branched or unbranched C₁₋₆alkyl. In a further preferred embodiment, R¹⁰ is hydrogen and R¹¹ is methyl. In another preferred embodiment, both R¹⁰ and R¹¹ are hydrogen. In certain embodiments, R¹² and R¹³ are branched or unbranched C₁₋₆alkyl, metal cation, or branched or unbranched C₁₋₆aralkyl. In certain preferred embodiments, R¹² and R¹³ are selected from benzyl, tert-butyl, and sodium cation. In more preferred embodiments, both R¹² and R¹³ are benzyl or tert-butyl. In the most preferred embodiment, at least one of R¹² and R¹³ is a sodium cation.

In some embodiments of PGPH activity inhibitors, any two of R¹, R², R³, and R⁴ are hydrogen and any two of R¹, R², R³, and R⁴ have a structure of formula (IIIa). In preferred embodiments any three of R¹, R², R³, and R⁴ are hydrogen and any one of R¹, R², R³, and R⁴ has a structure of formula (IIIa). In the most preferred embodiment, R¹ has a structure of formula (IIIa) and R², R³, and R⁴ are hydrogen.

In certain embodiments, R⁶ and R⁸ are branched or unbranched C₁₋₆ alkyl. In preferred embodiments, R⁶ and R⁸ are isobutyl. In preferred embodiments, R⁹ is hydrogen or acetyl. In the most preferred embodiments, R⁹ is acetyl. In a preferred embodiment, R¹⁰ is hydrogen and R¹¹ is methyl. In another preferred embodiment, both R¹⁰ and R¹¹ are hydrogen. In certain embodiments, R¹² and R¹³ are branched or unbranched C₁₋₆alkyl, metal cation, or branched or unbranched C₁₋₆aralkyl. In certain preferred embodiments, R¹² and R¹³ are selected from benzyl, tert-butyl, and sodium cation. In more preferred embodiments, both R¹² and R¹³ are benzyl or tert-butyl. In the most preferred embodiment, at least one of R¹² and R¹³ is a sodium cation.

Group 3

In another embodiment, the proteasome inhibitor has a structure of formula (V) or is a pharmaceutically acceptable salt thereof: 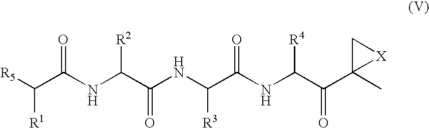
where:

each A is independently selected from C═O, C═S, and SO₂, preferably C═O;

L is absent or is selected from C═O, C═S, and SO₂, preferably L is absent or C═O;

M is absent or is C₁₋₈alkyl;

Q is absent or is selected from O, NH, and N—C₁₋₆alkyl, preferably Q is absent, O, or NH, most preferably Q is absent or O;

X is selected from O, NH, and N—C₁₋₆alkyl, preferably O;

Y is absent or is selected from O, NH, N—C₁₋₆alkyl, S, SO, SO₂, CHOR¹⁰, and CHCO₂R¹⁰;

each Z is independently selected from O, S, NH, and N—C₁₋₆alkyl, preferably O;

R¹, R², R³, and R⁴ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁵ is N(R⁶)LQR⁷;

R⁶ is selected from hydrogen, OH, and C₁₋₆alkyl, preferably C₁₋₆alkyl;

R⁷ is selected from hydrogen, C₁₋₆alkyl, C₁₋₆alkenyl, C₁₋₆alkynyl, aryl, C₁₋₆aralkyl, heteroaryl, C₁₋₆heteroaralkyl, R⁸ZA-C₁₋₈alkyl-, R¹¹Z-C₁₋₈alkyl-, (R⁸O)(R⁹O)P(═O)O—C₁₋₈alkyl-ZAZ-C₁₋₈alkyl-, (R⁸O)(R⁹O)P(═O)O—C₁₋₈alkyl-Z-C₁₋₈alkyl-, R⁸ZA-C₁₋₈alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH; or

R⁶ and R⁷ together are C₁₋₆alkyl-Y—C₁₋₆alkyl, C₁₋₆alkyl-ZA-C₁₋₆alkyl, A-C₁₋₆alkyl-ZA-C₁₋₆alkyl, A-C₁₋₆alkyl-A, or C₁₋₆alkyl-A, preferably C₁₋₂alkyl-Y—C₁₋₂alkyl, C₁₋₂alkyl-ZA-C₁₋₂alkyl, A-C₁₋₂alkyl-ZA-C₁₋₂alkyl, A-C₁₋₃alkyl-A, or C₁₋₄alkyl-A, thereby forming a ring;

R⁸ and R⁹ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R⁸ and R⁹ together are C₁₋₆alkyl, thereby forming a ring;

each R¹⁰ is independently selected from hydrogen and C₁₋₆alkyl, preferably C₁₋₆alkyl; and

R¹¹ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl,

provided that when R₆ is H, L is C═O, and Q is absent, R⁷ is not hydrogen, C₁₋₆alkyl, or substituted or unsubstituted aryl or heteroaryl.

In some embodiments, R¹, R², R³, and R⁴ are selected from C₁₋₆alkyl or C₁₋₆aralkyl. In preferred embodiments, R² and R⁴ are C₁₋₆alkyl and R¹ and R³ are C₁₋₆aralkyl. In the most preferred embodiment, R² and R⁴ are isobutyl, R¹ is 2-phenylethyl, and R³ is phenylmethyl.

In certain embodiments, L and Q are absent and R⁷ is selected from C₁₋₆alkyl, C₁₋₆alkenyl, C₁₋₆alkynyl, C₁₋₆aralkyl, and C₁₋₆heteroaralkyl. In certain such embodiments, R⁶ is C₁₋₆alkyl and R⁷ is selected from butyl, allyl, propargyl, phenylmethyl, 2-pyridyl, 3-pyridyl, and 4-pyridyl.

In other embodiments, L is SO₂, Q is absent, and R⁷ is selected from C₁₋₆alkyl and aryl. In certain such embodiments, R⁷ is selected from methyl and phenyl.

In certain embodiments, L is C═O and R⁷ is selected from C₁₋₆alkyl, C₁₋₆alkenyl, C₁₋₆alkynyl, aryl, C₁₋₆aralkyl, heteroaryl, C₁₋₆heteroaralkyl, R⁸ZA-C₁₋₈alkyl-, R¹¹Z-C₁₋₈alkyl-, (R⁸O)(R⁹O)P(═O)O—C₁₋₈alkyl-, (R⁸O)(R⁹O)P(═O)O—C₁₋₈alkyl-ZAZ-C₁₋₈alkyl-, (R⁸O)(R⁹O)P(═O)O—C₁₋₈alkyl-Z-C₁₋₈alkyl-, R⁸ZA-C₁₋₈alkyl-ZAZ-C₁₋₈alkyl-, heterocyclylMZAZ-C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH—. In certain embodiments, L is C═O, Q is absent, and R⁷ is H.

In certain embodiments, R⁶ is C₁₋₆alkyl, R⁷ is C₁₋₆alkyl, Q is absent, and L is C═O. In certain such embodiments, R⁷ is ethyl, isopropyl, 2,2,2-trifluoroethyl, or 2-(methylsulfonyl)ethyl.

In other embodiments, L is C═O, Q is absent, and R⁷ is C₁₋₆aralkyl. In certain such embodiments, R⁷ is selected from 2-phenylethyl, phenylmethyl, (4-methoxyphenyl)methyl, (4-chlorophenyl)methyl, and (4-fluorophenyl)methyl.

In other embodiments, L is C═O, Q is absent, R⁶ is C₁₋₆alkyl, and R⁷ is aryl. In certain such embodiments, R⁷ is substituted or unsubstituted phenyl.

In certain embodiments, L is C═O, Q is absent or O, n is 0 or 1, and R⁷ is —(CH₂)ₙcarbocyclyl. In certain such embodiments, R⁷ is cyclopropyl or cyclohexyl.

In certain embodiments, L and A are C═O, Q is absent, Z is O, n is an integer from 1 to 8 (preferably 1), and R⁷ is selected from R⁸ZA-C₁₋₈alkyl-, R¹¹Z-C₁₋₈alkyl-, R⁸ZA-C₁₋₈alkyl-ZAZ-C₁₋₈alkyl-, (R⁸O)(R⁹O)P(═O)O—C₁₋₈alkyl-ZAZ-C₁₋₈alkyl-, (R⁸O)(R⁹O)P(═O))—C₁₋₈alkyl-Z-C₁₋₈alkyl-, and heterocyclylMZAZ-C₁₋₈alkyl-. In certain such embodiments, R⁷ is heterocyclylMZAZ-C₁₋₈alkyl- where heterocyclyl is substituted or unsubstituted oxodioxolenyl or N(R¹²)(R¹³), wherein R¹² and R¹³ together are C₁₋₆alkyl-Y—C₁₋₆alkyl, preferably C₁₋₃alkyl-Y—C₁₋₃alkyl, thereby forming a ring.

In certain preferred embodiments, L is C═O, Q is absent, n is an integer from 1 to 8, and R⁷ is selected from (R⁸O)(R⁹O)P(═O)O—C₁₋₈alkyl-, (R¹⁰)₂NC₁₋₈alkyl, (R¹⁰)₃N⁺(CH₂)ₙ—, and heterocyclyl-M-. In certain such embodiments, R⁷ is —C₁₋₈alkylN(R¹⁰)₂ or —C₁₋₈alkylN⁺(R¹⁰)₃, where R¹⁰ is C₁₋₆alkyl. In certain other such embodiments, R⁷ is heterocyclylM-, where heterocyclyl is selected from morpholino, piperidino, piperazino, and pyrrolidino.

In certain embodiments, L is C═O, R⁶ is C₁₋₆alkyl, Q is selected from O and NH and R⁷ is selected from C₁₋₆alkyl, cycloalkyl-M, C₁₋₆araalkyl, and C₁₋₆heteroaraalkyl. In other embodiments, L is C═O, R⁶ is C₁₋₆alkyl, Q is selected from O and NH, and R⁷is C₁₋₆alkyl, where C₁₋₆alkyl is selected from methyl, ethyl, and isopropyl. In further embodiments, L is C═O, R⁶ is C₁₋₆alkyl, Q is selected from O and NH and R⁷ is C₁₋₆aralkyl, where aralkyl is phenylmethyl. In other embodiments, L is C═O, R⁶ is C₁₋₆alkyl, Q is selected from O and NH, and R⁷ is C₁₋₆heteroaraalkyl, where heteroaralkyl is (4-pyridyl)methyl.

In certain embodiments, L is absent or is C═O, and R⁶ and R⁷ together are C₁₋₆alkyl-Y—C₁₋₆alkyl, C₁₋₆alkyl-ZA-C₁₋₆alkyl, or C₁₋₆alkyl-A, thereby forming a ring. In certain preferred embodiments, L is C═O, Q and Y are absent, and R⁶ and R⁷ together are C₁₋₃alkyl-Y—C₁₋₃alkyl. In another preferred embodiment, L and Q are absent, and R⁶ and R⁷ together are C₁₋₃alkyl-Y—C₁₋₃alkyl. In another preferred embodiment, L is C═O, Q is absent, Y is selected from NH and N—C₁₋₆alkyl, and R⁶ and R⁷ together are C₁₋₃alkyl-Y—C₁₋₃alkyl. In another preferred embodiment, L is C═O, Y is absent, and R⁶ and R⁷ together are C₁₋₃alkyl-Y—C₁₋₃alkyl. In another preferred embodiment, L and A are C═O, and R⁶ and R⁷ together are C₁₋₂alkyl-ZA-C₁₋₂alkyl. In another preferred embodiment, L and A are C═O and R⁶and R⁷ together are C₂₋₃alkyl-A.

In certain embodiments, a compound of formula (V) has the following stereochemistry:

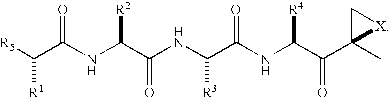

In preferred embodiments, the inhibitor has a structure of formula (VI) or a pharmaceutically acceptable salt thereof,

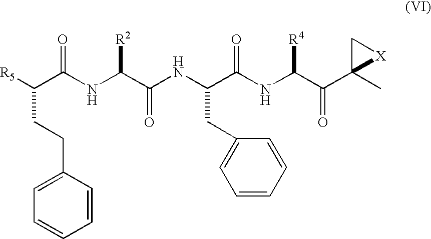

where:

each A is independently selected from C═O, C═S, and SO₂, preferably C═O;

L is absent or is selected from C═O, C═S, and SO₂, preferably L is absent or C═O;

M is absent or is C₁₋₈alkyl;

Q is absent or is selected from O, NH, and N—C₁₋₆alkyl, preferably Q is absent, O, or NH, most preferably Q is absent or O;

X is selected from O, NH, and N—C₁₋₆alkyl, preferably O;

Y is absent or is selected from O, NH, N—C₁₋₆alkyl, S, SO, SO₂, CHOR¹⁰, and CHCO₂R¹⁰;

each Z is independently selected from O, S, NH, and N—C₁₋₆alkyl, preferably O;

R² and R⁴ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁵ is N(R⁶)LQR⁷;

R⁶ is selected from hydrogen, OH, and C₁₋₆alkyl, preferably C₁₋₆alkyl;

R⁷is selected from hydrogen, C₁₋₆alkyl, C₁₋₆alkenyl, C₁₋₆alkynyl, aryl, C₁₋₆aralkyl, heteroaryl, C₁₋₆heteroaralkyl, R⁸ZA-C₁₋₈alkyl-, R¹¹Z-C₁₋₈alkyl-, (R⁸O)(R⁹O)P(═O)O—C₁₋₈alkyl-ZAZ-C₁₋₈alkyl-, (R⁸O)(R⁹O)P(═O)O—C₁₋₈alkyl-Z-C₁₋₈alkyl-, R⁸ZA-C₁₋₈alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH; or

R⁶ and R⁷ together are C₁₋₆alkyl-Y—C₁₋₆alkyl, C₁₋₆alkyl-ZA-C₁₋₆alkyl, A-C₁₋₆alkyl-ZA-C₁₋₆alkyl, A-C₁₋₆alkyl-A, or C₁₋₆alkyl-A, preferably C₁₋₂alkyl-Y—C₁₋₂alkyl, C₁₋₂alkyl-ZA-C₁₋₂alkyl, A-C₁₋₂alkyl-ZA-C₁₋₂alkyl, A-C₁₋₃alkyl-A, or C₁₋₄alkyl-A, thereby forming a ring;

R⁸ and R⁹ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R⁸ and R⁹ together are C₁₋₆alkyl, thereby forming a ring;

each R¹⁰ is independently selected from hydrogen and C₁₋₆alkyl, preferably C₁₋₆alkyl;

R¹¹ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl; and

provided that when R₆ is H, L is C═O, and Q is absent, R⁷ is not hydrogen, C₁₋₆alkyl, or substituted or unsubstituted aryl or heteroaryl.

In certain embodiments, L is C═O, Q is absent, X is O, R⁶ is H, and R² and R⁴ are selected from C₁₋₆alkyl and C₁₋₆aralkyl. In preferred such embodiments, R² and R⁴ are C₁₋₆alkyl. In the most preferred such embodiment, R² and R⁴ are isobutyl.

In certain embodiments, L is C═O, Q is absent, X is O, R⁶ is H, R² and R⁴ are isobutyl, and R⁷is heterocyclylM-, where the heterocycle is a nitrogen-containing heterocycle, such as piperazino (including N-(lower alkyl) piperazino), morpholino, and piperidino. In preferred such embodiments, M is CH₂. In the most preferred such embodiments, R⁷ is morpholino.

In certain embodiments, a compound of formula (VI) has the following structure, also referred to as peptide (a): 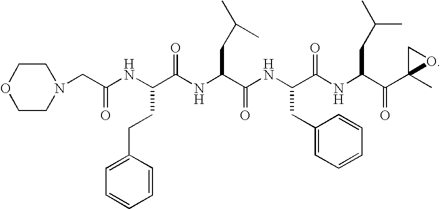
Goup 4

In a further embodiment, the proteasome inhibitors have a structure of formula (VII) or a pharmaceutically acceptable salt thereof,

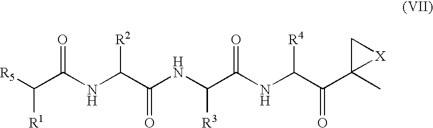

where:

each A is independently selected from C═O, C═S, and SO₂, preferably C═O;

each B is independently selected from C═O, C═S, and SO₂, preferably C═O;

D is absent or is C₁₋₈alkyl;

G is selected from O, NH, and N—C₁₋₆alkyl;

K is absent or is selected from C═O, C═S, and SO₂, preferably K is absent or is C═O;

L is absent or is selected from C═O, C═S, and SO₂, preferably L is absent or C═O;

M is absent or is C₁₋₈alkyl;

Q is absent or is selected from O, NH, and N—C₁₋₆alkyl, preferably Q is absent, O, or NH, most preferably Q is absent;

X is selected from O, S, NH, and N—C₁₋₆alkyl, preferably O;

each V is independently absent or is selected from O, S, NH, and N—C₁₋₆alkyl, preferably V is absent or O;

W is absent or is independently selected from O, S, NH, and N—C₁₋₆alkyl, preferably O;

Y is absent or is selected from O, NH, N—C₁₋₆alkyl, S, SO, SO₂, CHOR¹⁰, and CHCO₂R¹⁰;

each Z is independently selected from O, S, NH, and N—C₁₋₆alkyl, preferably O;

R¹, R², R³, and R⁴ are each independently selected from C₁₋₆alkyl, C₁₋₆hydroxyalkyl, C₁₋₆alkoxyalkyl, aryl, C₁₋₆aralkyl, and R¹⁴DVKOC₁₋₃alkyl-, wherein at least one of R¹ and R³ is R¹⁴DVKOC₁₋₃alkyl-;

R⁵ is N(R⁶)LQR⁷;

R⁶ is selected from hydrogen, OH, and C₁₋₆alkyl, preferably C₁₋₆alkyl;

R⁷ is a further chain of amino acids, hydrogen, a protecting group, aryl, or heteroaryl, any of which is optionally substituted with halogen, carbonyl, nitro, hydroxy, aryl, C₁₋₅alkyl; or R⁷ is selected from C₁₋₆alkyl, C₁₋₆alkenyl, C₁₋₆alkynyl, C₁₋₆aralkyl, C₁₋₆heteroaralkyl, R⁸ZA-C₁₋₈alkyl-, R¹¹Z-C₁₋₈alkyl-, (R⁸O)(R⁹O)P(═O)O—C₁₋₈alkyl-ZAZ-C₁₋₈alkyl-, (R⁸O)(R⁹O)P(═O)O—C₁₋₈alkyl-Z-C₁₋₈alkyl-, R⁸ZA-C₁₋₈alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH; or

R⁶ and R⁷ together are C₁₋₆alkyl-Y-C₁₋₆alkyl, C₁₋₆alkyl-ZA-C₁₋₆alkyl, A-C₁₋₆alkyl-ZA-C₁₋₆alkyl, A-C₁₋₆alkyl-A, or C₁₋₆alkyl-A, preferably C₁₋₂alkyl-Y—C₁₋₂alkyl, C₁₋₂alkyl-ZA-C₁₋₂alkyl, A-C₁₋₂alkyl-ZA-C₁₋₂alkyl, A-C₁₋₃alkyl-A, or C₁₋₄alkyl-A, thereby forming a ring, preferably R⁶ is hydrogen and R⁷ is C₁₋₆alkyl;

R⁸ and R⁹ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R⁸ and R⁹ together are C₁₋₆alkyl, thereby forming a ring;

each R¹⁰ is independently selected from hydrogen and C₁₋₆alkyl, preferably C₁₋₆alkyl;

R¹¹ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl,

R¹⁴ is selected from hydrogen, (R¹⁵O)(R¹⁶O)P((═O)W—, R¹⁵GB—, heterocyclyl-, (R¹⁷)₂N—, (R¹⁷)₃N⁺—, R¹⁷SO₂GBG-, and R¹⁵GBC₁₋₈alkyl- where the C₁₋₈alkyl moiety is optionally substituted with OH, C₁₋₈alkylW (optionally substituted with halogen, preferably fluorine), aryl, heteroaryl, carbocyclyl, heterocyclyl, and C₁₋₆aralkyl, preferably at least one occurrence of R¹⁴ is other than hydrogen;

R¹⁵ and R¹⁶ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R¹⁵ and R¹⁶ together are C₁₋₆alkyl, thereby forming a ring; and

R¹⁷ is selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl;

provided that when R₆ is H, L is C═O, and Q is absent, R⁷ is not hydrogen, C₁₋₆alkyl, or substituted or unsubstituted aryl or heteroaryl; and

D, G, V, K, and W are selected such that there are no O—O, N—O, S—N, or S—O bonds.

Suitable N-terminal protecting groups known in the art of peptide syntheses, include t-butoxy carbonyl (Boc), benzoyl (Bz), fluoren-9-ylmethoxycarbonyl (Fmoc), triphenylmethyl (trityl) and trichloroethoxycarbonyl (Troc) and the like. The use of various N-protecting groups, e.g., the benzyloxy carbonyl group or the t-butyloxycarbonyl group (Boc), various coupling reagents, e.g., dicyclohexylcarbodiimide (DCC), 1,3-diisopropylcarbodiimide (DIC), 1-(3-dimethylaminopropyl)-3-ethylcarbodiimide (EDC), N-hydroxyazabenzotriazole (HATU), carbonyldiimidazole, or 1-hydroxybenzotriazole monohydrate (HOBT), and various cleavage conditions: for example, trifluoroacetic acid (TFA), HCl in dioxane, hydrogenation on Pd—C in organic solvents (such as methanol or ethyl acetate), boron tris(trifluoroacetate), and cyanogen bromide, and reaction in solution with isolation and purification of intermediates are well-known in the art of peptide synthesis, and are equally applicable to the preparation of the subject compounds.

In certain embodiments, R¹, R², R³, and R⁴ are each independently selected from C₁₋₆alkyl, C₁₋₆hydroxyalkyl, C₁₋₆alkoxyalkyl, aryl, C₁₋₆aralkyl, and R¹⁴DVKOC₁₋₃alkyl- wherein at least one of R¹ and R³ is R¹⁴DVKOC₁₋₃alkyl-. In preferred embodiments, one of R¹ and R³ is C₁₋₆aralkyl and the other is R¹⁴DVKOC₁₋₃alkyl-, and R² and R⁴ are independently C₁₋₆alkyl. In the most preferred embodiment, one of R¹ and R³ is 2-phenylethyl or phenylmethyl and the other is R¹⁴DVKOCH₂— or R¹⁴DVKO(CH₃)CH—, and both R² and R⁴ are isobutyl.

In certain embodiments, L and Q are absent and R⁷ is selected from hydrogen, a further chain of amino acids, C₁₋₆acyl, a protecting group, aryl, heteroaryl, C₁₋₆alkyl, C₁₋₆alkenyl, C₁₋₆alkynyl, C₁₋₆aralkyl, and C₁₋₆heteroaralkyl. In certain such embodiments, R⁶ is C₁₋₆alkyl and R⁷ is selected from butyl, allyl, propargyl, phenylmethyl, 2-pyridyl, 3-pyridyl, and 4-pyridyl.

In other embodiments, L is SO₂, Q is absent, and R⁷ is selected from C₁₋₆alkyl and aryl. In certain such embodiments, R⁷ is selected from methyl and phenyl.

In certain embodiments, L is C═O and R⁷ is selected from C₁₋₆alkyl, C₁₋₆alkenyl, C₁₋₆alkynyl, aryl, C₁₋₆aralkyl, heteroaryl, C₁₋₆heteroaralkyl, R⁸ZA-C₁₋₈alkyl-, R¹¹Z-C₁₋₈alkyl-, (R⁸O)(R⁹O)P(═O)O—C₁₋₈alkyl-, (R⁸O)(R⁹O)P(═O)O—C₁₋₈alkyl-ZAZ-C₁₋₈alkyl-, (R⁸O)(R⁹O)P(═O)O—C₁₋₈alkyl-Z-C₁₋₈alkyl-, R⁸ZA-C₁₋₈alkyl-ZAZ-C₁₋₈alkyl-, heterocyclylMZAZ-C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH—. In certain embodiments, L is C═O, Q is absent, and R⁷ is H.

In certain embodiments, R⁶is C₁₋₆alkyl, R⁷is C₁₋₆alkyl, Q is absent, and L is C═O. In certain such embodiments, R⁷ is ethyl, isopropyl, 2,2,2-trifluoroethyl, or 2-(methylsulfonyl)ethyl.

In other embodiments, L is C═O, Q is absent, and R⁷ is C₁₋₆aralkyl. In certain such embodiments, R⁷ is selected from 2-phenylethyl, phenylmethyl, (4-methoxyphenyl)methyl, (4-chlorophenyl)methyl, and (4-fluorophenyl)methyl.

In other embodiments, L is C═O, Q is absent, R⁶ is C₁₋₆alkyl, and R⁷ is aryl. In certain such embodiments, R⁷ is substituted or unsubstituted phenyl.

In certain embodiments, L is C═O, Q is absent or O, and R⁷ is —(CH₂)ₙcarbocyclyl. In certain such embodiments, R⁷ is cyclopropyl or cyclohexyl.

In certain embodiments, L and A are C═O, Q is absent, Z is O, and R⁷is selected from R⁸ZA-C₁₋₈alkyl-, R¹¹Z-C₁₋₈alkyl-, R⁸ZA-C₁₋₈alkyl-ZAZ-C₁₋₈alkyl-, (R⁸O)(R⁹O)P(═O)O—C₁₋₈alkyl-ZAZ-C₁₋₈alkyl-, (R⁸O)(R⁹O)P(═O)O—C₁₋₈alkyl-Z-C₁₋₈alkyl-, and heterocyclylMZAZ-C₁₋₈alkyl-. In certain such embodiments, R⁷is heterocyclylMZAZ-C₁₋₈alkyl- where heterocyclyl is substituted or unsubstituted oxodioxolenyl or N(R¹²)(R¹³), wherein R¹² and R¹³ together are C₁₋₆alkyl-Y—C₁₋₆alkyl, preferably C₁₋₃alkyl-Y—C₁₋₃alkyl, thereby forming a ring.

In certain preferred embodiments, L is C═O, Q is absent, and R⁷is selected from (R⁸O)(R⁹O)P(═O)O—C₁₋₈alkyl-, (R¹⁰)₂NC₁₋₈alkyl, (R¹⁰)₃N⁺(CH₂)ₙ—, and heterocyclyl-M-. In certain such embodiments, R⁷ is —C₁₋₈alkylN(R¹⁰)₂ or —C₁₋₈alkylN⁺(R¹⁰)₃, where R¹⁰ is C₁₋₆alkyl. In certain other such embodiments, R⁷is heterocyclylM-, where heterocyclyl is selected from morpholino, piperidino, piperazino, and pyrrolidino.

In certain embodiments, L is C═O, R⁶ is C₁₋₆alkyl, Q is selected from O and NH and R⁷ is selected from C₁₋₆alkyl, cycloalkyl-M, C₁₋₆araalkyl, and C₁₋₆heteroaraalkyl. In other embodiments, L is C═O, R⁶ is C₁₋₆alkyl, Q is selected from O and NH, and R⁷is C₁₋₆alkyl, where C₁₋₆alkyl is selected from methyl, ethyl, and isopropyl. In further embodiments, L is C═O, R⁶ is C₁₋₆alkyl, Q is selected from O and NH and R⁷is C₁₋₆aralkyl, where aralkyl is phenylmethyl. In other embodiments, L is C═O, R⁶ is C₁₋₆alkyl, Q is selected from O and NH, and R⁷is C₁₋₆heteroaralkyl, where heteroaralkyl is (4-pyridyl)methyl.

In certain embodiments, L is absent or is C═O, and R⁶ and R⁷ together are C₁₋₆alkyl-Y—C₁₋₆alkyl, C₁₋₆alkyl-ZA-C₁₋₆alkyl, or C₁₋₆alkyl-A, thereby forming a ring. In certain preferred embodiments, L is C═O, Q and Y are absent, and R⁶ and R⁷ together are C₁₋₃alkyl-Y—C₁₋₃alkyl. In another preferred embodiment, L and Q are absent, and R⁶ and R⁷ together are C₁₋₃alkyl-Y—C₁₋₃alkyl. In another preferred embodiment, L is C═O, Q is absent, Y is selected from NH and N—C₁₋₆alkyl, and R⁶ and R⁷ together are C₁₋₃alkyl-Y—C₁₋₃alkyl. In another preferred embodiment, L is C═O, Y is absent, and R⁶ and R⁷together are C₁₋₃alkyl-Y—C₁₋₃alkyl. In another preferred embodiment, L and A are C═O, and R⁶ and R⁷ together are C₁₋₂alkyl-ZA-C₁₋₂alkyl. In another preferred embodiment, L and A are C═O and R⁶ and R⁷ together are C₂₋₃alkyl-A.

In certain embodiments, R¹⁴ is (R¹⁵O)(R¹⁶O)P(═O)W—. In certain such embodiments, D, V, K, and W are absent. In other such embodiments, V and K are absent, D is C₁₋₈alkyl, and W is O. In yet other such embodiments, D is C₁₋₈alkyl, K is C═O, and V and W are O.

In certain embodiments, R¹⁴ is R¹⁵GB—. In preferred embodiments, B is C═O, G is O, D is C₁₋₈alkyl, V is O, and K is C═O.

In certain embodiments, R¹⁴ is heterocyclyl-. In preferred such embodiments, D is C₁₋₈alkyl. In certain such embodiments, V is O, K is C═O, and heterocyclyl is oxodioxolenyl. In other such embodiments, V is absent, K is absent or is C═O, and heterocyclyl is N(R¹⁸)(R¹⁹), where R¹⁸ and R¹⁹ together are J-T-J, J-WB-J, or B-J-T-J, T is absent or is selected from O, NR¹⁷, S, SO, SO₂, CHOR¹⁷, CHCO₂R¹⁵, C═O, CF₂, and CHF, and J is absent or is C₁₋₃alkyl.

In certain embodiments, R¹⁴is (R¹⁷)₂N— or (R¹⁷)₃N⁺—, and preferably V is absent. In preferred such embodiments, D is C₁₋₈alkyl and K is absent or C═O. In certain embodiments where V is absent and R¹⁴ is (R¹⁷)₂N—, D is absent K is absent or is C═O, preferably K is C═O.

In certain embodiments, R¹⁴ is R¹⁷SO₂GBG-. In preferred such embodiments, B is C═O, D, V, and K are absent, and G is NH or NC₁₋₆alkyl.

In certain embodiments, R¹⁴ is R¹⁵GBC₁₋₈alkyl-. In preferred embodiments, B is C═O, G is O, and the C₁₋₈alkyl moiety is optionally substituted with OH, C₁₋₈alkyl (optionally substituted with halogen, preferably fluorine), C₁₋₈alkylW, aryl, heteroaryl, carbocyclyl, heterocyclyl, and C₁₋₆aralkyl. In certain such embodiments, the C₁₋₈alkyl moiety is an unsubstituted, mono-, or disubstituted C₁alkyl.

In certain embodiments, a compound of formula (VII) has the following stereochemistry:

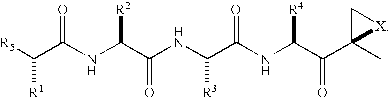

In preferred embodiments, the inhibitor has a structure of formula (VIII) or a pharmaceutically acceptable salt thereof,

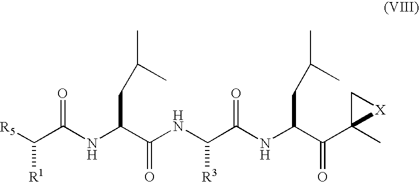

where:

each A is independently selected from C═O, C═S, and SO₂, preferably C═O;

each B is independently selected from C═O, C═S, and SO₂, preferably C═O;

D is absent or is C₁₋₈alkyl;

G is selected from O, NH, and N—C₁₋₆alkyl;

K is absent or is selected from C═O, C═S, and SO₂, preferably K is absent or is C═O;

L is absent or is selected from C═O, C═S, and SO₂, preferably L is absent or C═O;

M is absent or is C₁₋₈alkyl;

Q is absent or is selected from O, NH, and N—C₁₋₆alkyl, preferably Q is absent, O, or NH, most preferably Q is absent or O;

X is selected from O, S, NH, and N—C₁₋₆alkyl, preferably O;

each V is independently absent or is selected from O, S, NH, and N—C₁₋₆alkyl, preferably V is absent or O;

W is absent or is independently selected from O, S, NH, and N—C₁₋₆alkyl, preferably O;

Y is absent or is selected from O, NH, N—C₁₋₆alkyl, S, SO, SO₂, CHOR¹⁰, and CHCO₂R¹⁰;

each Z is independently selected from O, S, NH, and N—C₁₋₆alkyl, preferably O;

R¹ and R³ are each independently selected from C₁₋₆alkyl, C₁₋₆hydroxyalkyl, C₁₋₆alkoxyalkyl, aryl, C₁₋₆aralkyl, and R¹⁴DVKOC₁₋₃alkyl-, wherein at least one of R¹ and R³ is R¹⁴DVKOC₁₋₃alkyl-;

R⁵ is N(R⁶)LQR⁷;

R⁶ is selected from hydrogen, OH, and C₁₋₆alkyl, preferably C₁₋₆alkyl;

R⁷ is a further chain of amino acids, hydrogen, a protecting group, aryl, or heteroaryl, any of which is optionally substituted with halogen, carbonyl, nitro, hydroxy, aryl, C₁₋₅alkyl; or R⁷ is selected from C₁₋₆alkyl, C₁₋₆alkenyl, C₁₋₆alkynyl, C₁₋₆aralkyl, C₁₋₆heteroaralkyl, R⁸ZA-C₁₋₈alkyl-, R¹¹Z-C₁₋₈alkyl-, (R⁸O)(R⁹O)P(═O)O—C₁₋₈alkyl-ZAZ-C₁₋₈alkyl-, (R⁸O)(R⁹O)P(═O)O—C₁₋₈alkyl-Z-C₁₋₈alkyl-, R⁸ZA-C₁₋₈alkyl-ZAZ-C₁₋₈alkyl-, heterocyclylMZAZ-C₁₋₈alkyl-, (R⁸O)(R⁹O)P(═O)O—C₁₋₈alkyl-, (R¹⁰)₂N—C₁₋₈alkyl-, (R¹⁰)₃N⁺—C₁₋₈alkyl-, heterocyclylM-, carbocyclylM-, R¹¹SO₂C₁₋₈alkyl-, and R¹¹SO₂NH; or

R⁶ and R⁷ together are C₁₋₆alkyl-Y—C₁₋₆alkyl, C₁₋₆alkyl-ZA-C₁₋₆alkyl, A-C₁₋₆alkyl-ZA-C₁₋₆alkyl, A-C₁₋₆alkyl-A, or C₁₋₆alkyl-A, preferably C₁₋₂alkyl-Y—C₁₋₂alkyl, C₁₋₂alkyl-ZA-C₁₋₂alkyl, A-C₁₋₂alkyl-ZA-C₁₋₂alkyl, A-C₁₋₃alkyl-A, or C₁₋₄alkyl-A, thereby forming a ring;

R⁸ and R⁹ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R⁸ and R⁹ together are C₁₋₆alkyl, thereby forming a ring;

each R¹⁰ is independently selected from hydrogen and C₁₋₆alkyl, preferably C₁₋₆alkyl; and

R¹¹ is independently selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl,

R¹⁴ is selected from hydrogen, (R¹⁵O)(R¹⁶O)P(═O)W—, R¹⁵GB—, heterocyclyl-, (R¹⁷)₂N—, (R¹⁷)₃N⁺—, R¹⁷SO₂GBG-, and R¹⁵GBC₁₋₈alkyl- where the C₁₋₈alkyl moiety is optionally substituted with OH, C₁₋₈alkylW (optionally substituted with halogen, preferably fluorine), aryl, heteroaryl, carbocyclyl, heterocyclyl, and C₁₋₆aralkyl, preferably at least one occurrence of R¹⁴ is other than hydrogen;

R¹⁵ and R¹⁶ are independently selected from hydrogen, metal cation, C₁₋₆alkyl, C₁₋₆alkenyl, C₁₋₆alkynyl, aryl, heteroaryl, C₁₋₆aralkyl, and C₁₋₆heteroaralkyl, preferably from hydrogen, metal cation, and C₁₋₆alkyl, or R¹⁵ and R¹⁶ together are C₁₋₆alkyl, thereby forming a ring;

R¹⁷ is selected from hydrogen, C₁₋₆alkyl, C₁₋₆alkenyl, C₁₋₆alkynyl, carbocyclyl, heterocyclyl, aryl, heteroaryl, C₁₋₆aralkyl, and C₁₋₆heteroaralkyl;

provided that when R₆ is H, L is C═O, and Q is absent, R⁷ is not hydrogen, C₁₋₆alkyl, or substituted or unsubstituted aryl or heteroaryl; and

D, G, V, K, and W are selected such that there are no O—O, N—O, S—N, or S—O bonds.

Group 5

In one embodiment, the proteasome inhibitors have a structure of formula (IX) or a pharmaceutically acceptable salt thereof,

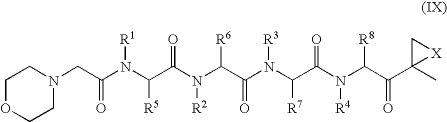

where:

X is O, NH, or N-alkyl, preferably O;

R¹, R², R³, and R⁴ are independently selected from hydrogen and a group of formula (IXa), preferably, R¹, R², R³, and R⁴ are all the same, more preferably R¹, R², R³, and R⁴ are all hydrogen;

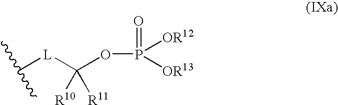

R⁵, R⁶, R⁷, and R⁸ are independently selected from H, C₁₋₆alkyl, C₁₋₆hydroxyalkyl, C₁₋₆alkoxyalkyl, aryl, and C₁₋₆ aralkyl, each of which is optionally substituted with a group selected from amide, amine, carboxylic acid or a pharmaceutically acceptable salt thereof, carboxyl ester, thiol, and thioether, preferably R⁵, R⁶, R⁷, and R⁸ are independently selected from C₁₋₆alkyl, C₁₋₆hydroxyalkyl, and C₁₋₆aralkyl, more preferably, R⁶ and R⁸ are independently C₁₋₆alkyl and R⁵ and R⁷ are independently C₁₋₆aralkyl;

R¹⁰ and R¹¹ are independently selected from hydrogen and C₁₋₆alkyl, or R¹⁰ and R¹¹ together form a 3- to 6-membered carbocyclic or heterocyclic ring;

R¹² and R¹³ are independently selected from hydrogen, a metal cation, C₁₋₆alkyl, and C₁₋₆aralkyl, or R¹² and R¹³ together represent C₁₋₆alkyl, thereby forming a ring.

In certain embodiments, X is O and R¹, R², R³, and R⁴ are all the same, preferably R¹, R², R³, and R⁴ are all hydrogen. In certain such embodiments, R⁵, R⁶, R⁷, and R⁸ are independently selected from C₁₋₆alkyl, C₁₋₆hydroxyalkyl, and C₁₋₆aralkyl, more preferably, R⁶ and R⁸ are independently C₁₋₆alkyl and R⁵ and R⁷ are independently C₁₋₆aralkyl.

In certain preferred embodiments, X is O, R¹, R², R³, and R⁴ are all hydrogen, R⁶ and R⁸ are both isobutyl, R⁵ is phenylethyl, and R⁷ is phenylmethyl.

In certain embodiments, a compound of formula (IX) has the following stereochemistry:

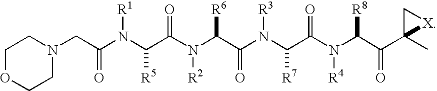

In preferred embodiments, the inhibitor has a structure of formula (X) or a pharmaceutically acceptable salt thereof,

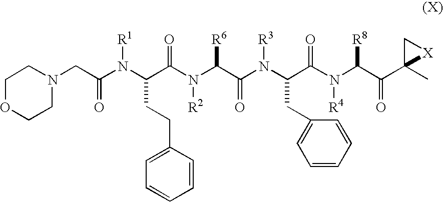

where:

X is O, NH, or N-alkyl, preferably O;

R¹, R², R³, and R⁴ are independently selected from hydrogen and a group of formula (IXa), preferably, R¹, R², R³, and R⁴ are all the same, more preferably R¹, R², R³, and R⁴ are all hydrogen;

R⁶ and R⁸ are independently selected from H, C₁₋₆alkyl, C₁₋₆hydroxyalkyl, C₁₋₆alkoxyalkyl, aryl, and C₁₋₆aralkyl, each of which is optionally substituted with a group selected from amide, amine, carboxylic acid or a pharmaceutically acceptable salt thereof, carboxyl ester, thiol, and thioether, preferably R⁶ and R⁸ are independently selected from C₁₋₆alkyl, C₁₋₆hydroxyalkyl, and C₁₋₆aralkyl, more preferably, R⁶ and R⁸ are independently C₁₋₆alkyl.

In certain embodiments, X is O and R¹, R², R³, and R⁴ are all the same, preferably R¹, R², R³, and R⁴ are all hydrogen. In certain such embodiments, R⁶ and R⁸ are independently selected from C₁₋₆alkyl, C₁₋₆hydroxyalkyl, and C₁₋₆aralkyl, more preferably, R⁶ and R⁸ are independently C₁₋₆alkyl.

In certain preferred embodiments, X is O, R¹, R², R³, and R⁴ are all hydrogen, and R⁶ and R⁸ are both isobutyl.

In certain embodiments, a compound of formula (X) has the following structure: 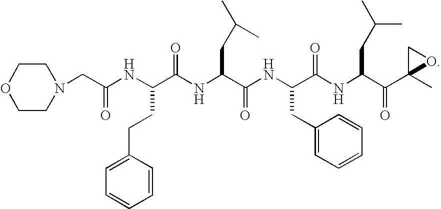
Group 6

In certain embodiments, the proteasome inhibitors have a structure of formula (XI) or a pharmaceutically acceptable salt thereof,

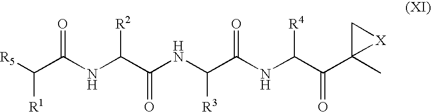

where:

X is selected from O, NH, and N—C₁₋₆alkyl, preferably O;

R¹, R², R³, and R⁴ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁵ is N(R⁶)R⁷;

R⁶ is selected from hydrogen, OH, and C₁₋₆alkyl, preferably H or C₁₋₆alkyl; and

R⁷ is a detectable label, such as a fluorescent moiety, a chemiluminescent moiety, a paramagnetic contrast agent, a metal chelate, a radioactive isotope-containing moiety (e.g., a moiety containing one or more tritium atoms), biotin, or a moiety that selectively binds to an antibody.

In some embodiments, R¹, R², R³, and R⁴ are selected from C₁₋₆alkyl or C₁₋₆aralkyl. In preferred embodiments, R² and R⁴ are C₁₋₆alkyl and R¹ and R³ are C₁₋₆aralkyl. In the most preferred embodiment, R² and R⁴ are isobutyl, R¹ is 2-phenylethyl, and R³ is phenylmethyl.

In certain embodiments, R⁶ is selected from H or C₁₋₆alkyl. In certain preferred embodiments, R⁶ is H.

In certain embodiments, R⁷ is a covalently conjugated moiety selected from a fluorescent moiety, a radioactive isotope-containing moiety, biotin, and a moiety that selectively binds to an antibody.

In certain embodiments, R⁷ is a fluorescent moiety. In certain such embodiments, the fluorescent moiety is an amine-reactive dye that has been covalently attached to the inhibitor. In preferred such embodiments, the amine-reactive dye is selected from Alexa Fluor dyes, BODIPY dyes, Cascade Blue dyes, coumarin, digoxigenin, fluorescein, lissamine rhodamine B dyes, Oregon Green dyes, rhodamine 6G dyes, rhodamine green dyes, rhodamine red dyes, Tamra, tetramethylrhodamine, and Texas Red dyes. In certain preferred embodiments, R⁷ is a fluorescent moiety selected from fluorescein, tetramethylrhodamine, and Tamra.

There are generally four classes of commonly used dye reagents to label amines: succinimidyl esters, isothiocyanates, sulfonyl chlorides, and tetrafluorophenyl esters. Generally succinimidyl esters and tetrafluorophenyl esters are preferred for conjugation to proteins and peptides since they form a stable amide bond between the dye and the protein. Useful reviews that provide information on the conjugation of an amine-reactive dye to a protein or peptide sequence can be found in Bioconjug. Chem. 3, 2 (1992) and Methods Mol. Biol. 45, 205 (1995), incorporated herein by reference in their entirety. Information on the purchase and use of amine-reactive dyes is also available from Molecular Probes, Inc.

In certain embodiments, R⁷ contains a radioactive moiety. In certain such embodiments, R⁷ is selected from C₁₋₆alkyl, C₁₋₆hydroxyalkyl, C₁₋₆alkoxyalkyl, aryl, and C₁₋₆aralkyl, wherein R⁷ includes at least one radioactive label selected from ³H, ¹¹C, ¹⁴C, ¹³N, and ¹⁵O. In preferred such embodiments, R⁷ is an amino acid or peptide moiety that includes at least one radioactive label selected from ¹¹C, ¹⁴C, ¹³N, and ¹⁵O.

In certain embodiments, R⁷ is a covalently conjugated moiety that selectively binds to an antibody. In preferred embodiments, the moiety is selected from FLAG™, HA, HIS, c-Myc, VSV-G, V5 and HSV.

Preparation of inhibitors where R⁷ is selected from FLAG™, HA, HIS, c-Myc, VSV-G, V5 and HSV may be accomplished using standard peptide coupling chemistry.

In certain embodiments, R⁷ is biotin which may be covalently conjugated to the inhibitor using standard carboxylic acid/amine coupling chemistry.

In certain embodiments, a compound of formula (XI) has the following stereochemistry:

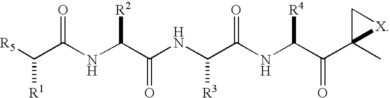

In preferred embodiments, the inhibitor has a structure of formula (XII) or a pharmaceutically acceptable salt thereof, 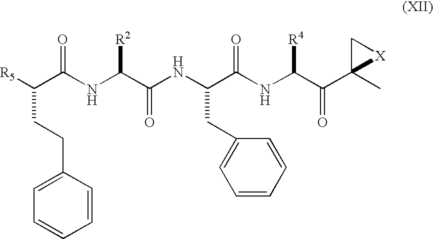
where:

X is selected from O, NH, and N—C₁₋₆alkyl, preferably O;

R² and R⁴ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁵ is N(R⁶)R⁷;

R⁶ is selected from hydrogen, OH, and C₁₋₆alkyl, preferably C₁₋₆alkyl;

R⁷ is a detectable label, such as a fluorescent moiety, a chemiluminescent moiety, a paramagnetic contrast agent, a metal chelate, a radioactive isotope-containing moiety, biotin, or a moiety that selectively binds to an antibody.

Group 7

In certain embodiments, the proteasome inhibitors have a structure of formula (XIII) or formula (XIV) or a pharmaceutically acceptable salt thereof, 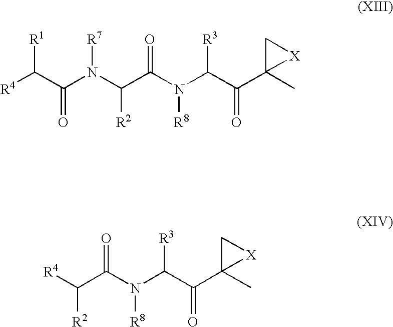
where:

each Ar is independently an aromatic or heteroaromatic group optionally substituted with 1 to 4 substituents;

L is selected from C═O, C═S, and SO₂, preferably SO₂ or C═O;

X is selected from O, S, NH, and N—C₁₋₆alkyl, preferably O;

Y is absent or is selected from C═O and SO₂;

Z is absent or is C₁₋₆alkyl;

R¹, R², and R³ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁴ is N(R⁵)L-Z-R⁶;

R⁵ is selected from hydrogen, OH, C₁₋₆aralkyl, and C₁₋₆alkyl, preferably hydrogen;

R⁶ is selected from hydrogen, C₁₋₆alkenyl, Ar—Y—, carbocyclyl, and heterocyclyl; and

R⁷ and R⁸ are independently selected from hydrogen, C₁₋₆alkyl, and C₁₋₆aralkyl, preferably hydrogen.

In certain embodiments, X is O and R¹, R², and R³ are each independently selected from C₁₋₆alkyl, C₁₋₆hydroxyalkyl, and C₁₋₆aralkyl. In preferred such embodiments, R¹ and R³ are independently C₁₋₆alkyl and R² is C₁₋₆aralkyl. In more preferred such embodiments, R¹ and R³ are both isobutyl and R² is phenylmethyl.

In certain embodiments, R⁵ is hydrogen, L is C═O or SO₂, R⁶ is Ar—Y—, and each Ar is independently selected from phenyl, indolyl, benzofuranyl, naphthyl, quinolinyl, quinolonyl, thienyl, pyridyl, pyrazyl, and the like. In certain such embodiments, Ar may be substituted with Ar-Q-, where Q is selected from a direct bond, —O—, and C₁₋₆alkyl. In certain other such embodiments where Z is C₁₋₆alkyl, Z may be substituted, preferably with Ar, e.g., phenyl.

In certain embodiments, R⁵ is hydrogen, Z is absent, L is C═O or SO₂, and R⁶ is selected from Ar—Y and heterocyclyl. In certain preferred such embodiments, heterocyclyl is selected from chromonyl, chromanyl, morpholino, and piperidinyl. In certain other preferred such embodiments, Ar is selected from phenyl, indolyl, benzofuranyl, naphthyl, quinolinyl, quinolonyl, thienyl, pyridyl, pyrazyl, and the like.

In certain embodiments, R⁵ is hydrogen, L is C═O or SO₂, Z is absent, and R⁶ is C₁₋₆alkenyl, where C₁₋₆alkenyl is a substituted vinyl group where the substituent is preferably an aryl or heteroaryl group, more preferably a phenyl group optionally substituted with one to four substituents.

In certain embodiments, R⁷ and R⁸ are independently selected from hydrogen and C₁₋₆alkyl. In certain preferred such embodiments, R⁷ and R⁸ are independently selected from hydrogen and methyl. In more preferred such embodiments, R⁷ and R⁸ are both hydrogen.

In certain embodiments, a compound of formula (XIII) or formula (XIV) has the following stereochemistry

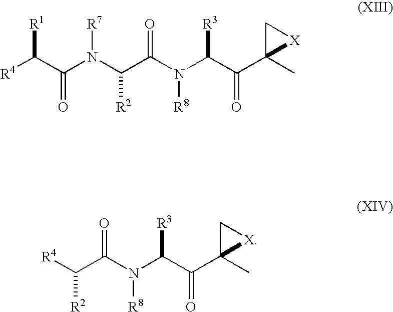

In preferred embodiments, the inhibitor has a structure of formula (XV) or formula (XVI) or a pharmaceutically acceptable salt thereof: 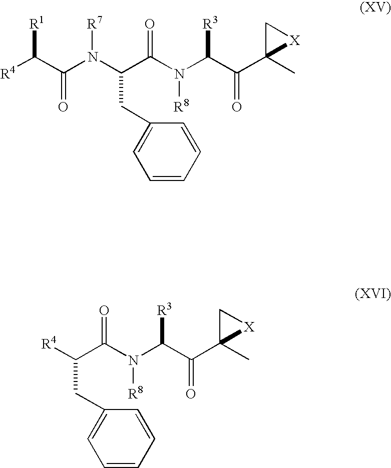
where:

each Ar is independently an aromatic or heteroaromatic group optionally substituted with 1-4 substituents;

L is selected from C═O, C═S, and SO₂, preferably SO₂ or C═O;

X is selected from O, S, NH, and N—C₁₋₆alkyl, preferably O;

Y is absent or is selected from C═O and SO₂;

Z is absent or is C₁₋₆alkyl;

R¹ and R³ are each independently selected from C₁₋₆alkyl, C₁₋₆hydroxyalkyl, C₁₋₆alkoxyalkyl, aryl, and C₁₋₆aralkyl, any of which is optionally substituted with one or more of amide, amine, carboxylic acid (or a salt thereof), ester (including C₁₋₅ alkyl ester and aryl ester), thiol, or thioether substituents;

R⁴ is N(R⁵)L-Z-R⁶;

R⁵ is selected from hydrogen, OH, C₁₋₆aralkyl, and C₁₋₆alkyl, preferably hydrogen;

R⁶ is selected from hydrogen, C₁₋₆alkenyl, Ar—Y—, carbocyclyl, and heterocyclyl; and

R⁷ and R⁸ are independently selected from hydrogen, C₁₋₆alkyl, and C₁₋₆aralkyl, preferably hydrogen.

In certain embodiments, X is O and R¹ and R³ are each independently selected from C₁₋₆alkyl, C₁₋₆hydroxyalkyl, and C₁₋₆aralkyl. In preferred such embodiments, R¹ and R³ are independently C₁₋₆alkyl. In more preferred such embodiments, R¹ and R³ are isobutyl.

In certain embodiments, R⁵ is hydrogen, L is C═O or SO₂, and R⁶is Ar—Y—, each Ar is independently selected from phenyl, indolyl, benzofuranyl, naphthyl, quinolinyl, quinolonyl, thienyl, pyridyl, pyrazyl, and the like. In certain such embodiments, Ar may be substituted with Ar-Q-, where Q is selected from a direct bond, —O—, and C₁₋₆alkyl. In certain other such embodiments where Z is C₁₋₆alkyl, Z may be substituted, e.g., preferably with Ar, more preferably with phenyl.

In certain embodiments, R⁵ is hydrogen, Z is absent, L is C═O or SO₂, and R⁶ is selected from Ar—Y and heterocyclyl. In certain preferred such embodiments, heterocyclyl is selected from chromonyl, chromanyl, morpholino, and piperidinyl. In certain other preferred such embodiments Ar is selected from phenyl, indolyl, benzofuranyl, naphthyl, quinolinyl, quinolonyl, thienyl, pyridyl, pyrazyl, and the like.

In certain embodiments, R⁵ is hydrogen, L is C═O or SO₂, Z is absent, and R⁶ is C₁₋₆alkenyl, where C₁₋₆alkenyl is a substituted vinyl group where the substituent is preferably an aryl or heteroaryl group, more preferably the substituent is a phenyl group optionally substituted with one to four substituents.

In certain embodiments, R⁷ and R⁸ are independently selected from hydrogen and C₁₋₆alkyl. In certain preferred such embodiments, R⁷ and R⁸ are independently selected from hydrogen and methyl. In more preferred such embodiments, R⁷ and R⁸ are both hydrogen.

In certain embodiments, -L-Z-R⁶ is selected from

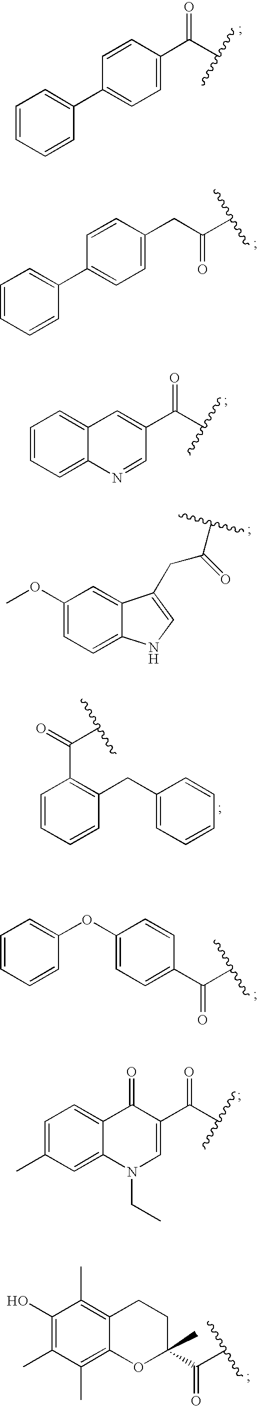

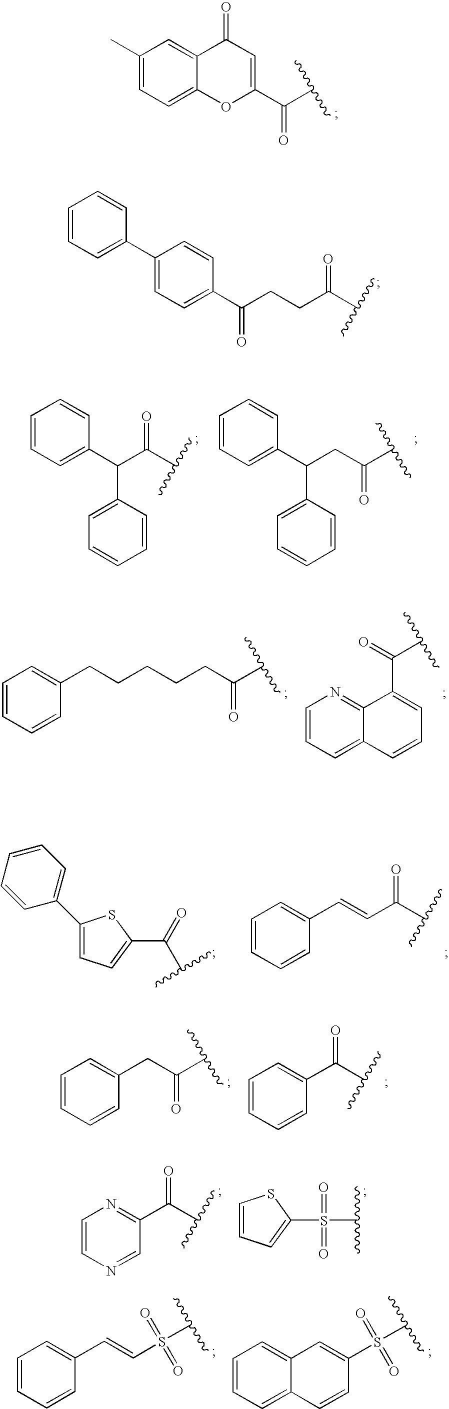

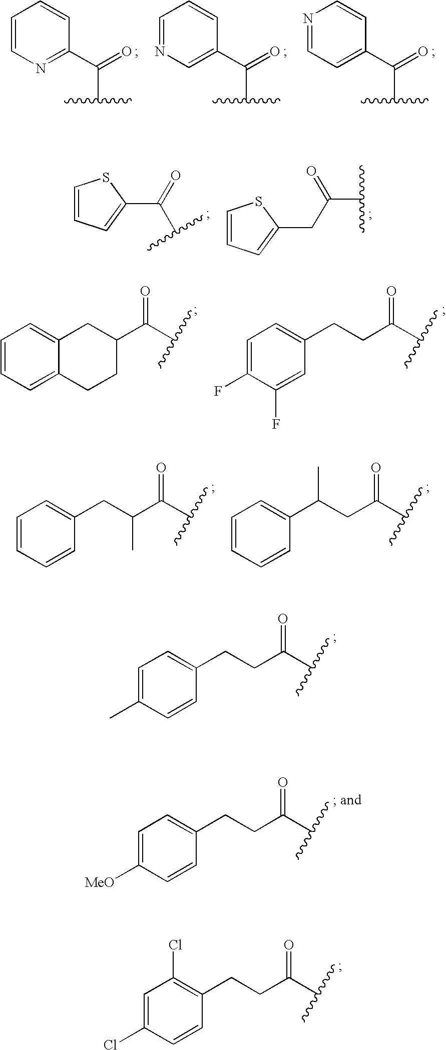

In embodiments including such groups bonded to α′ carbons, the stereochemistry of the α′-carbon (that carbon forming a part of the epoxide or aziridine ring) can be (R) or (S). The invention is based, in part, on the structure-function information disclosed herein, which suggests the following preferred stereochemical relationships. Note that a preferred compound may have a number of stereocenters having the indicated up-down (or β-α, where β as drawn herein is above the plane of the page) or (R)—(S) relationship (that is, it is not required that every stereocenter in the compound conform to the preferences stated). In some preferred embodiments, the stereochemistry of the α′ carbon is (R), that is, the X atom is β, or above the plane of the molecule.

In certain embodiments inhibitors of the invention may have asymmetric centers that may have either (R) or (S) stereochemistry. The invention is based, in part, on the structure-function information disclosed herein, which suggests the following preferred stereochemical relationships. Note that a preferred compound may have a number of stereocenters having the indicated up-down (or β-α, where β as drawn herein is above the plane of the page) or (R)—(S) relationship (that is, it is not required that every stereocenter in the compound conform to the preferences stated). In some preferred embodiments, the stereochemistry of the α′ carbon is (R), that is, the X atom is β, or above the plane of the molecule.

Other Proteasome Inhibitors

The administration regimens of the invention are equally applicable to other types of proteasome inhibitors, including boronic acids such as bortezomib, lactacystin derivatives, C-terminal peptidyl aldehydes, peptide vinyl sulfones, and other peptide boronates. Descriptions of proteasome inhibitors useful in the present methods may be found in U.S. Pat. Nos. 6,849,743, 6,794,516, 6,781,000, 6,740,674, 6,656,904, 6,660,268, 6,294,560, 6,133,308, and 6,075,150, and in US published patent applications Nos. 20050025734, 20040254118, 20040171556, 20040138153, 20040116329, 20040106539, 20040097420, 20020111292, 20020107203, and 20020103127, all of which are hereby incorporated by reference in their entirety. Any of these compounds may be advantageously administered using the methods disclosed herein.

### DEFINITIONS

The term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkoxy” refers to an alkyl group having an oxygen attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “C₁₋₆alkoxyalkyl” refers to a C₁₋₆alkyl group substituted with an alkoxy group, thereby forming an ether.

The term “C₁₋₆aralkyl”, as used herein, refers to a C₁₋₆alkyl group substituted with an aryl group.

The terms “amine” and “amino” are art-recognized and refer to both unsubstituted and substituted amines and salts thereof, e.g., a moiety that can be represented by the general formulae: 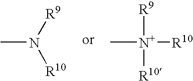
where R⁹, R¹⁰ and R^{10′} each independently represent a hydrogen, an alkyl, an alkenyl, —(CH₂)ₘ—R⁸, or R⁹ and R¹⁰ taken together with the N atom to which they are attached complete a heterocycle having from 4 to 8 atoms in the ring structure; R⁸ represents an aryl, a cycloalkyl, a cycloalkenyl, a heterocyclyl or a polycyclyl; and m is zero or an integer from 1 to 8. In preferred embodiments, only one of R⁹ or R¹⁰ can be a carbonyl, e.g., R⁹, R¹⁰, and the nitrogen together do not form an imide. In even more preferred embodiments, R⁹ and R¹⁰ (and optionally R^{10′}) each independently represent a hydrogen, an alkyl, an alkenyl, or —(CH₂)ₘ—R⁸. In certain embodiments, an amino group is basic, meaning its protonated form has a pKₐ above 7.00.

The terms “amide” and “amido” are art-recognized as an amino-substituted carbonyl and includes a moiety that can be represented by the general formula: 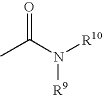
wherein R⁹, R¹⁰ are as defined above. Preferred embodiments of the amide will not include imides which may be unstable.

The term “aryl” as used herein includes 5-, 6-, and 7-membered substituted or unsubstituted single-ring aromatic groups in which each atom of the ring is carbon. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “buffer” is a substance which by its presence in solution increases the amount of acid or alkali that must be added to cause unit change in pH. Thus, a buffer is a substance that assists in regulating the pH of a composition. Typically, a buffer is chosen based upon the desired pH and compatibility with other components of a composition. In general, a buffer has a pKₐ that is no more than 1 unit less than or greater than the desired pH of the composition (or that the composition will produce upon dissolution).

The terms “carbocycle” and “carbocyclyl”, as used herein, refer to a non-aromatic substituted or unsubstituted ring in which each atom of the ring is carbon. The terms “carbocycle” and “carbocyclyl” also include polycyclic ring systems having two or more cyclic rings in which two or more carbons are common to two adjoining rings wherein at least one of the rings is carbocyclic, e.g., the other cyclic rings can be cycloalkyls, cycloalkenyls, cycloalkynyls, aryls, heteroaryls, and/or heterocyclyls.

The term “carbonyl” is art-recognized and includes such moieties as can be represented by the general formula: 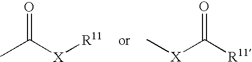
wherein X is a bond or represents an oxygen or a sulfur, and R¹¹ represents a hydrogen, an alkyl, an alkenyl, —(CH₂)ₘ—R⁸ or a pharmaceutically acceptable salt, R^{11′} represents a hydrogen, an alkyl, an alkenyl or —(CH₂)ₘ—R⁸, where m and R⁸ are as defined above. Where X is an oxygen and R¹¹ or R^{11′} is not hydrogen, the formula represents an “ester”. Where X is an oxygen, and R¹¹ is a hydrogen, the formula represents a “carboxylic acid”.

As used herein, “enzyme” can be any partially or wholly proteinaceous molecule which carries out a chemical reaction in a catalytic manner. Such enzymes can be native enzymes, fusion enzymes, proenzymes, apoenzymes, denatured enzymes, farnesylated enzymes, ubiquitinated enzymes, fatty acylated enzymes, gerangeranylated enzymes, GPI-linked enzymes, lipid-linked enzymes, prenylated enzymes, naturally-occurring or artificially-generated mutant enzymes, enzymes with side chain or backbone modifications, enzymes having leader sequences, and enzymes complexed with non-proteinaceous material, such as proteoglycans, proteoliposomes. Enzymes can be made by any means, including natural expression, promoted expression, cloning, various solution-based and solid-based peptide syntheses, and similar methods known to those of skill in the art.

The term “C₁₋₆heteroaralkyl”, as used herein, refers to a C₁₋₆alkyl group substituted with a heteroaryl group.

The term “heteroaryl” includes substituted or unsubstituted aromatic 5- to 7-membered ring structures, more preferably 5- to 6-membered rings, whose ring structures include one to four heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, triazole, pyrazole, pyridine, pyrazine, pyridazine and pyrimidine, and the like.

The term “heteroatom” as used herein means an atom of any element other than carbon or hydrogen. Preferred heteroatoms are nitrogen, oxygen, phosphorus, and sulfur.

The term “heterocyclyl” or “heterocyclic group” refers to substituted or unsubstituted non-aromatic 3- to 10-membered ring structures, more preferably 3- to 7-membered rings, whose ring structures include one to four heteroatoms. The term “heterocyclyl” or “heterocyclic group” also includes polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “C₁₋₆hydroxyalkyl” refers to a C₁₋₆alkyl group substituted with a hydroxy group.

As used herein, the term “inhibitor” is meant to describe a compound that blocks or reduces an activity of an enzyme (for example, inhibition of proteolytic cleavage of standard fluorogenic peptide substrates such as suc-LLVY-AMC, Box-LLR-AMC and Z-LLE-AMC, inhibition of various catalytic activities of the 20S proteasome). An inhibitor can act with competitive, uncompetitive, or noncompetitive inhibition. An inhibitor can bind reversibly or irreversibly, and therefore the term includes compounds that are suicide substrates of an enzyme. An inhibitor can modify one or more sites on or near the active site of the enzyme, or it can cause a conformational change elsewhere on the enzyme.

As used herein, the term “peptide” includes not only standard amide linkage with standard et-substituents, but commonly utilized peptidomimetics, other modified linkages, non-naturally occurring side chains, and side chain modifications, as detailed herein.

The terms “polycyclyl” or “polycyclic” refer to two or more rings (e.g., cycloalkyls, cycloalkenyls, cycloalkynyls, aryls, heteroaryls, and/or heterocyclyls) in which two or more carbons are common to two adjoining rings, e.g., the rings are “fused rings”. Each of the rings of the polycycle can be substituted or unsubstituted.

The term “practically insoluble” refers to proteasome inhibitors that generally have a solubility of less than 0.1 mg/mL in water. The invention also encompasses proteasome inhibitors having a water solubility of less than 0.05 mg/mL and even less than 0.01 mg/mL.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

A “therapeutically effective amount” of a compound with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “thioether” refers to an alkyl group, as defined above, having a sulfur moiety attached thereto. In preferred embodiments, the “thioether” is represented by —S— alkyl. Representative thioether groups include methylthio, ethylthio, and the like.

As used herein, the term “treating” or “treatment” includes reversing, reducing, or arresting the symptoms, clinical signs, and underlying pathology of a condition in manner to improve or stabilize a subject's condition.

Uses for Proteasome Inhibitor Compositions

The biological consequences of proteasome inhibition are numerous. Proteasome inhibition has been suggested as a prevention and/or treatment of a multitude of diseases including, but not limited to, proliferative diseases, neurotoxic/degenerative diseases, Alzheimer's, ischemic conditions, inflammation, immune-related diseases, HIV, cancers, organ graft rejection, septic shock, inhibition of antigen presentation, decreasing viral gene expression, parasitic infections, conditions associated with acidosis, macular degeneration, pulmonary conditions, muscle wasting diseases, fibrotic diseases, bone and hair growth diseases. Therefore, dosing strategies for administering pharmaceutical formulations of proteasome inhibitor compositions, such as the epoxy ketone class of molecules, provide a means of treating patients with these conditions.

Proteasome inhibitor compositions can be used to treat conditions mediated directly by the proteolytic function of the proteasome such as muscle wasting, or mediated indirectly via proteins which are processed by the proteasome such as NF-κB. The proteasome participates in the rapid elimination and post-translational processing of proteins (e.g., enzymes) involved in cellular regulation (e.g., cell cycle, gene transcription, and metabolic pathways), intercellular communication, and the immune response (e.g., antigen presentation). Specific examples discussed below include β-amyloid protein and regulatory proteins such as cyclins and transcription factor NF-κB.

At the cellular level, the accumulation of polyubiquitinated proteins, cell morphological changes, and apoptosis have been reported upon treatment of cells with various proteasome inhibitors. The proteasome degrades many proteins in maturing reticulocytes and growing fibroblasts. In cells deprived of insulin or serum, the rate of proteolysis nearly doubles. Inhibiting the proteasome reduces proteolysis, thereby reducing both muscle protein loss and the nitrogenous load on kidneys or liver. One embodiment of the invention relates to the treatment of cachexia and muscle-wasting diseases. Methods of the invention are useful for treating conditions such as cancer, chronic infectious diseases, fever, muscle disuse (atrophy) and denervation, nerve injury, fasting, renal failure associated with acidosis, and hepatic failure. See, e.g., Goldberg, U.S. Pat. No. 5,340,736. Embodiments of the invention therefore encompass methods for: reducing the rate of muscle protein degradation in a cell; reducing the rate of intracellular protein degradation; reducing the rate of degradation of p53 protein in a cell; and inhibiting the growth of p53-related cancers. Each of these methods includes contacting a cell (in vivo or in vitro, e.g., a muscle in a subject) with an effective amount of a pharmaceutical composition comprising a proteasome inhibitor in a manner disclosed herein.

Intracellular proteolysis generates small peptides for presentation to T-lymphocytes to induce MHC class I-mediated immune responses. The immune system screens for autologous cells that are virally infected or have undergone oncogenic transformation. One embodiment is a method for inhibiting antigen presentation in a cell, including exposing the cell to a compound described herein. A dosing strategy for proteasome inhibitors of the invention may be used to treat immune-related conditions such as allergy, asthma, organ/tissue rejection (graft-versus-host disease), and auto-immune diseases, including, but not limited to, lupus, rheumatoid arthritis, psoriasis, multiple sclerosis, and inflammatory bowel diseases (such as ulcerative colitis and Crohn's disease). Thus, a further embodiment is a method for suppressing the immune system of a subject including administering to the subject an effective amount of a proteasome inhibitor compound in a manner described herein.

Another further embodiment is a method for altering the repertoire of antigenic peptides produced by the proteasome or other Ntn with multicatalytic activity. For example, if the PGPH activity of 20S proteasome is selectively inhibited, a different set of antigenic peptides will be produced by the proteasome and presented in MHC molecules on the surfaces of cells than would be produced and presented either without any enzyme inhibition, or with, for example, selective inhibition of chymotrypsin-like activity of the proteasome.

Another embodiment of the invention is the use of proteasome inhibitor compositions administered in a manner disclosed herein for the treatment of neurodegenerative diseases and conditions, including, but not limited to, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis).

Alzheimer's disease is characterized by extracellular deposits of β-amyloid protein (β-AP) in senile plaques and cerebral vessels. β-AP is a peptide fragment of 39 to 42 amino acids derived from an amyloid protein precursor (APP). At least three isoforms of APP are known (695, 751, and 770 amino acids). Alternative splicing of mRNA generates the isoforms; normal processing affects a portion of the β-AP sequence, thereby preventing the generation of β-AP. It is believed that abnormal protein processing by the proteasome contributes to the abundance of β-AP in the Alzheimer brain. The APP-processing enzyme in rats contains about ten different subunits (22 kDa-32 kDa). The 25 kDa subunit has an N-terminal sequence of X-Gln-Asn-Pro-Met-X-Thr-Gly-Thr-Ser, which is identical to the β-subunit of human macropain (Kojima, S. et al., Fed. Eur. Biochem. Soc., (1992) 304:57-60). The APP-processing enzyme cleaves at the Gln¹⁵-Lys¹⁶ bond; in the presence of calcium ion, the enzyme also cleaves at the Met-¹-Asp¹ bond, and the Asp¹-Ala² bonds to release the extracellular domain of β-AP.

One embodiment, therefore, is a method of treating Alzheimer's disease, including administering to a subject an effective amount of a proteasome inhibitor composition in a manner disclosed herein. Such treatment includes reducing the rate of β-AP processing, reducing the rate of β-AP plaque formation, reducing the rate of β-AP generation, and reducing the clinical signs of Alzheimer's disease.

Fibrosis is the excessive and persistent formation of scar tissue resulting from the hyperproliferative growth of fibroblasts and is associated with activation of the TGF-β signaling pathway. Fibrosis involves extensive deposition of extracellular matrix and can occur within virtually any tissue or across several different tissues. Normally, the level of intracellular signaling protein (Smad) that activate transcription of target genes upon TGF-β stimulation is regulated by proteasome activity (Xu et al., 2000). However, accelerated degradation of the TGF-β signaling components has been observed in cancers and other hyperproliferative conditions. Thus, certain embodiments of the invention relate to a method for treating hyperproliferative conditions such as diabetic retinopathy, macular degeneration, diabetic nephropathy, glomerulosclerosis, IgA nephropathy, cirrhosis, biliary atresia, congestive heart failure, scleroderma, radiation-induced fibrosis, and lung fibrosis (idiopathic pulmonary fibrosis, collagen vascular disease, sarcoidosis, interstitial lung diseases and extrinsic lung disorders). The treatment of burn victims is often hampered by fibrosis, thus, an additional embodiment of the invention is the topical or systemic administration of the inhibitors to treat burns. Wound closure following surgery is often associated with disfiguring scars, which may be prevented by inhibition of fibrosis. Thus, in certain embodiments, the invention relates to a method for the prevention or reduction of scarring.

Certain proteasome inhibitors block both degradation and processing of ubiquitinated NF-κB in vitro and in vivo. Proteasome inhibitors also block IκB-A degradation and NF-κB activation (Palombella, et al. Cell (1994) 78:773-785; and Traenckner, et al., EMBO J. (1994) 13:5433-5441). One embodiment of the invention is a method for inhibiting IκB-α degradation, including contacting the cell with a compound described herein. A further embodiment is a method for reducing the cellular content of NF-κB in a cell, muscle, organ, or subject, including contacting the cell, muscle, organ, or subject with a proteasome inhibitor compound in the manner described herein.

NF-κB is a member of the Rel protein family. The Rel family of transcriptional activator proteins can be divided into two groups. The first group requires proteolytic processing, and includes p50 (NF-κB1, 105 kDa) and p52 (NF-κ2, 100 kDa). The second group does not require proteolytic processing, and includes p65 (RelA, Rel (c-Rel), and RelB). Both homo- and heterodimers can be formed by Rel family members; NF-κB, for example, is a p50-p65 heterodimer. After phosphorylation and ubiquitination of IκB and p105, the two proteins are degraded and processed, respectively, to produce active NF-κB which translocates from the cytoplasm to the nucleus. Ubiquitinated p105 is also processed by purified proteasomes (Palombella et al., Cell (1994) 78:773-785). Active NF-κB forms a stereospecific enhancer complex with other transcriptional activators and, e.g., HMG I(Y), inducing selective expression of a particular gene.

NF-κB regulates genes involved in the immune and inflammatory response, and mitotic events. For example, NF-κB is required for the expression of the immunoglobulin light chain κ gene, the IL-2 receptor α-chain gene, the class I major histocompatibility complex gene, and a number of cytokine genes encoding, for example, IL-2, IL-6, granulocyte colony-stimulating factor, and IFN-β (Palombella et al., Cell (1994) 78:773-785). Some embodiments of the invention include methods of affecting the level of expression of IL-2, MHC-I, IL-6, TNFα, IFN-β or any of the other previously-mentioned proteins, each method including administering to a subject an effective amount of a proteasome inhibitor composition in a manner disclosed herein. Complexes including p50 are rapid mediators of acute inflammatory and immune responses (Thanos, D. and Maniatis, T., Cell (1995) 80:529-532).

Overproduction of lipopolysaccharide (LPS)-induced cytokines such as TNFα is considered to be central to the processes associated with septic shock. Furthermore, it is generally accepted that the first step in the activation of cells by LPS is the binding of LPS to specific membrane receptors. The α- and β-subunits of the 20S proteasome complex have been identified as LPS-binding proteins, suggesting that the LPS-induced signal transduction may be an important therapeutic target in the treatment or prevention of sepsis (Qureshi, N. et al., J. Immun. (2003) 171: 1515-1525). Therefore, in certain embodiments, the methods of administering proteasome inhibitor compositions may be used for the inhibition of TNFα to prevent and/or treat septic shock.

NF-κB also participates in the expression of the cell adhesion genes that encode E-selectin, P-selectin, ICAM, and VCAM-1 (Collins, T., Lab. Invest (1993) 68:499-508). One embodiment of the invention is a method for inhibiting cell adhesion (e.g., cell adhesion mediated by E-selectin, P-selectin, ICAM, or VCAM-1), including contacting a cell with (or administering to a subject) an effective amount of a pharmaceutical composition comprising a proteasome inhibitor in a manner disclosed herein.

NF-κB also binds specifically to the HIV-enhancer/promoter. When compared to the Nef of mac239, the HIV regulatory protein Nef of pbj14 differs by two amino acids in the region which controls protein kinase binding. It is believed that the protein kinase signals the phosphorylation of IκB, triggering IκB degradation through the ubiquitin-proteasome pathway. After degradation, NF-κB is released into the nucleus, thus enhancing the transcription of HIV (Cohen, J., Science, (1995) 267:960). Two embodiments of the invention are a method for inhibiting or reducing HIV infection in a subject, and a method for decreasing the level of viral gene expression, each method including administering to the subject an effective amount of a proteasome inhibitor composition in a manner disclosed herein.

Ischemia and reperfusion injury results in hypoxia, a condition in which there is a deficiency of oxygen reaching the tissues of the body. This condition causes increased degradation of Iκ-Bα, thereby resulting in the activation of NF-κB (Koong et al., 1994). It has been demonstrated that the severity of injury resulting in hypoxia can be reduced with the administration of a proteasome inhibitor (Gao et al., 2000; Bao et al., 2001; Pye et al., 2003). Therefore, certain embodiments of the invention relate to a method of treating an ischemic condition or reperfusion injury comprising administering to a subject in need of such treatment an effective amount of a proteasome inhibitor compound in a manner disclosed herein. Examples of such conditions or injuries include, but are not limited to, acute coronary syndrome (vulnerable plaques), arterial occlusive disease (cardiac, cerebral, peripheral arterial and vascular occlusions), atherosclerosis (coronary sclerosis, coronary artery disease), infarctions, heart failure, pancreatitis, myocardial hypertrophy, stenosis, and restenosis.

Other eukaryotic transcription factors that require proteolytic processing include the general transcription factor TFIIA, herpes simplex virus VP16 accessory protein (host cell factor), virus-inducible IFN regulatory factor 2 protein, and the membrane-bound sterol regulatory element-binding protein 1.

Other embodiments of the invention are methods for affecting cyclin-dependent eukaryotic cell cycles, including exposing a cell (in vitro or in vivo) to a proteasome inhibitor composition in a manner disclosed herein. Cyclins are proteins involved in cell cycle control. The proteasome participates in the degradation of cyclins. Examples of cyclins include mitotic cyclins, G1 cyclins, and cyclin B. Degradation of cyclins enables a cell to exit one cell cycle stage (e.g., mitosis) and enter another (e.g., division). It is believed all cyclins are associated with p34^{cdc2} protein kinase or related kinases. The proteolysis targeting signal is localized to amino acids 42-RAALGNISEN-50 (destruction box). There is evidence that cyclin is converted to a form vulnerable to a ubiquitin ligase or that a cyclin-specific ligase is activated during mitosis (Ciechanover, A., Cell, (1994) 79:13-21). Inhibition of the proteasome inhibits cyclin degradation, and therefore inhibits cell proliferation, for example, in cyclin-related cancers (Kumatori et al., Proc. Natl. Acad. Sci. USA (1990) 87:7071-7075). One embodiment of the invention is a method for treating a proliferative disease in a subject (e.g., cancer, psoriasis, or restenosis), including administering to the subject an effective amount of a proteasome inhibitor composition in a manner disclosed herein. The invention also encompasses a method for treating cyclin-related inflammation in a subject, including adminstering to a subject a therapeutically effective amount of a proteasome inhibitor composition in a manner described herein.

Additional embodiments are methods for affecting the proteasome-dependent regulation of oncoproteins and methods of treating or inhibiting cancer growth, each method including exposing a cell (in vivo, e.g., in a subject, or in vitro) to a proteasome inhibitor composition in a manner disclosed herein. HPV-16 and HPV-18-derived E6 proteins stimulate ATP-and ubiquitin-dependent conjugation and degradation of p53 in crude reticulocyte lysates. The recessive oncogene p53 has been shown to accumulate at the nonpermissive temperature in a cell line with a mutated thermolabile El. Elevated levels of p53 may lead to apoptosis. Examples of proto-oncoproteins degraded by the ubiquitin system include c-Mos, c-Fos, and c-Jun. One embodiment is a method for treating p53-related apoptosis, including administering to a subject an effective amount of a proteasome inhibitor composition in a manner disclosed herein.

In another embodiment, the disclosed compositions are useful for the treatment of a parasitic infection, such as infections caused by protozoan parasites. The proteasome of these parasites is considered to be involved primarily in cell differentiation and replication activities (Paugam et al., Trends Parasitol. 2003, 19(2): 55-59). Furthermore, entamoeba species have been shown to lose encystation capacity when exposed to proteasome inhibitors (Gonzales, et al., Arch. Med. Res. 1997, 28, Spec No: 139-140). In certain such embodiments, the administrative protocols for the proteasome inhibitor compositions are useful for the treatment of parasitic infections in humans caused by a protozoan parasite selected from Plasmodium sps. (including P. falciparum, P. vivax, P. malariae, and P. ovale, which cause malaria), Trypanosoma sps. (including T. cruzi, which causes Chagas' disease, and T. brucei which causes African sleeping sickness), Leishmania sps. (including L. amazonesis, L. donovani, L. infantum, L. mexicana, etc.), Pneumocystis carinii (a protozoan known to cause pneumonia in AIDS and other immunosuppressed patients), Toxoplasma gondii, Entamoeba histolytica, Entamoeba invadens, and Giardia lamblia. In certain embodiments, the disclosed administrative strategies for proteasome inhibitor compositions are useful for the treatment of parasitic infections in animals and livestock caused by a protozoan parasite selected from Plasmodium hermani, Cryptosporidium sps., Echinococcus granulosus, Eimeria tenella, Sarcocystis neurona, and Neurospora crassa. Other compounds useful as proteasome inhibitors in the treatment of parasitic diseases are described in WO 98/10779, which is incorporated herein in its entirety.

In certain embodiments, the dosing strategy of proteasome inhibitor compositions inhibit proteasome activity in a parasite without recovery in red blood cells and white blood cells. In certain such embodiments, the long half-life of blood cells may provide prolonged protection with regard to therapy against recurring exposures to parasites. In certain embodiments, the administrative protocol for proteasome inhibitor compositions may provide prolonged protection with regard to chemoprophylaxis against future infection.

It has also been demonstrated that inhibitors that bind to the 20S proteasome stimulate bone formation in bone organ cultures. Furthermore, when such inhibitors have been administered systemically to mice, certain proteasome inhibitors increased bone volume and bone formation rates over 70% (Garrett, I. R. et al., J. Clin. Invest. (2003) 111: 1771-1782), therefore suggesting that the ubiquitin-proteasome machinery regulates osteoblast differentiation and bone formation. Therefore, the disclosed methods for administering proteasome inhibitor compositions may be useful in the treatment and/or prevention of diseases associated with bone loss, such as osteroporosis.

Proteasome inhibition has already been validated as a therapeutic strategy for the treatment of cancer, particularly multiple myeloma. However, based on both in vitro and in vivo models, one would predict that it could serve as a strategy against other cancers, particularly heme-related malignancies and solid tumors. Therefore, certain embodiments of the invention relate to a method of treating cancers comprising administering to a subject in need of such treatment an effective amount of a proteasome inhibitor compound in a manner disclosed herein.

Administration

The precise time of administration and/or amount of the proteasome inhibitor composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

Along with dosage variation depending on the symptoms, age and body weight of the patient, the nature and severity of the disorder to be treated or prevented, the route of administration and the form of the drug can also vary. For example, where the proteasome inhibitor compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent in addition to a cyclodextrin and a buffer.

In certain embodiments of the invention, the route of administration and form of the proteasome inhibitor is the same for both the first dose and the second dose of proteasome inhibitor. In another embodiment, the route of administration and form of the second dose of proteasome inhibitor is different than that of the first dose of proteasome inhibitor. A further embodiment of the invention is the administration of a third dose of proteasome inhibitor that may or may not be similar in form or route of administration as the second dose of proteasome inhibitor.

The amount of active ingredient which can be combined with a carrier material to produce a single dosage form will generally be that amount of the compound which produces a therapeutic effect. In certain embodiments of the invention the amount of active ingredient in the second dose of proteasome inhibitor is equal to the amount of active ingredient of the first dose. In another embodiment of the invention, the amount of active ingredient in the second dose of proteasome inhibitor is different from that of the first dose. In some instances less active ingredient may be desired in the second dose, whereas in other circumstances, more active ingredient may be desired in the second dose of proteasome inhibitor. A further embodiment of the invention is a third dose of proteasome inhibitor that may or may not comprise the same amount of active ingredient as the second dose of proteasome inhibitor.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salt” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In other cases, the inhibitors useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylaamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of an inhibitor(s) as an active ingredient. A composition may also be administered as a bolus, electuary, or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, cyclodextrins, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to inhibitor(s), excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to an inhibitor(s), excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

The proteasome inhibitor(s) can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of an inhibitor(s) to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more inhibitors(s) in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of agents may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

These inhibitors(s) may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the inhibitor(s), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The concentration of a disclosed compound in a pharmaceutically acceptable mixture will vary depending on several factors, including the dosage of the compound to be administered, the pharmacokinetic characteristics of the compound(s) employed, and the route of administration

In certain embodiments, a pharmaceutical formulation as described above may be designed to release drug slowly over a period of time, e.g., sustained- or controlled-release delivery. In this way, proteasome activity may be subject to more constant inhibition, reducing the magnitude of the suppression-recovery variation in activity that occurs using immediate-release formulations. With such formulations, it may be possible to achieve the effect of the methods disclosed herein while still separating administration of doses of proteasome inhibitor by more than 24 hours, more than 48 hours, or even more than 72 hours. In some cases, it may be possible to achieve suppression of proteasome activity by administering such sustained release formulations several days, a week, or even weeks apart.

Additional approaches exist to achieve substantially continuous administration of a drug such as a proteasome inhibitor. For example, an inhibitor may be administered by an IV drip, a drug delivery pump (such as pumps currently used for administration of insulin, or devices such as those made by BioValve (e.g., the e-Patch™)), a microneedle patch (such as the Micro-Trans™ device made by BioValve), or other devices designed to administer a drug gradually over an extended period, whether by continuous delivery from an external source, or repeated application of devices that deliver the drug over a relatively short period of time.

Another aspect of the invention provides a conjoint therapy wherein one or more other therapeutic agents are administered with the protease inhibitor. Such conjoint treatment may be achieved by way of the simultaneous, sequential, or separate dosing of the individual components of the treatment.

In certain embodiments, a proteasome inhibitor is conjointly administered with one or more other proteasome inhibitor(s).

In certain embodiments, a chemotherapeutic is conjointly administered with a dosage of proteasome inhibitor. Suitable chemotherapeutics may include, natural products such as vinca alkaloids (i.e., vinblastine, vincristine, and vinorelbine), paclitaxel, epidipodophyllotoxins (i.e., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates (busulfan), nitrosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine); aromatase inhibitors (anastrozole, exemestane, and letrozole); and platinum coordination complexes (cisplatin, carboplatin), procarbazine, hydroxyurea, mitotane, aminoglutethimide; histone deacetylase (HDAC) inhibitors (trichostatin, sodium butyrate, apicidan, suberoyl anilide hydroamic acid); hormones (i.e. estrogen) and hormone agonists such as leutinizing hormone releasing hormone (LHRH) agonists (goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, a dosage of proteasome inhibitor is conjointly administered with a cytokine. Cytokines include, but are not limited to, Interferon-γ, -α, and -β, Interleukins 1-8, 10 and 12, Granulocyte Monocyte Colony-Stimulating factor (GM-CSF), TNF-α and -β, and TGF-β.

In certain embodiments, a dose of proteasome inhibitor of the invention is conjointly administered with a steroid. Suitable steroids may include, but are not limited to, 21-acetoxypregnenolone, aldlometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof.

In certain embodiments, a dose of proteasome inhibitor of the invention is conjointly administered with an immunotherapeutic agent. Suitable immunotherapeutic agents may include, but are not limited to, MDR modulators (verapamil, valspodar, biricodar, tariquidar, laniquidar), rapamycin, mycophenylate mofetil, cyclophosomide, cyclosporine, thalidomide, and monoclonal antibodies. The monoclonal antibodies can be either naked or conjugated such as rituximab, tositumomab, alemtuzumab, daclizumab epratuzumab, ibritumomab tiuxetan, gemtuzumab ozogamicin, bevacizumab, cetuximab, erlotinib and trastuzumab.

### EXEMPLIFICATION

To demonstrate that increasing the amount of time that proteasome activity is inhibited correlates to increased cytotoxicity, and thereby decreased cell viability, the following experiments were performed.

Prolonging the Time of Exposure to the Inhibitor Leads to Increased Cytotoxicity.

Cells were exposed to Compound A for various lengths of time. At the end of the exposure time, cells were washed to remove any drug that remained in the media. 72 hours post exposure initiation, a cell viability assay was performed. The assay was performed using three types of cell lines: Non-transformed (non-tumor), Heme tumor lines and Solid tumor lines. Based on the data shown in FIG. 2, differential cytotoxic effects could be seen based on the exposure time of the cells to the inhibitor.

Suppressing Recovery of Proteasome Activity, Leading to Increased Cytoxicity and Decreased Cell Viability, Can Be Achieved By Increasing the Frequency of Inhibitor Exposure.

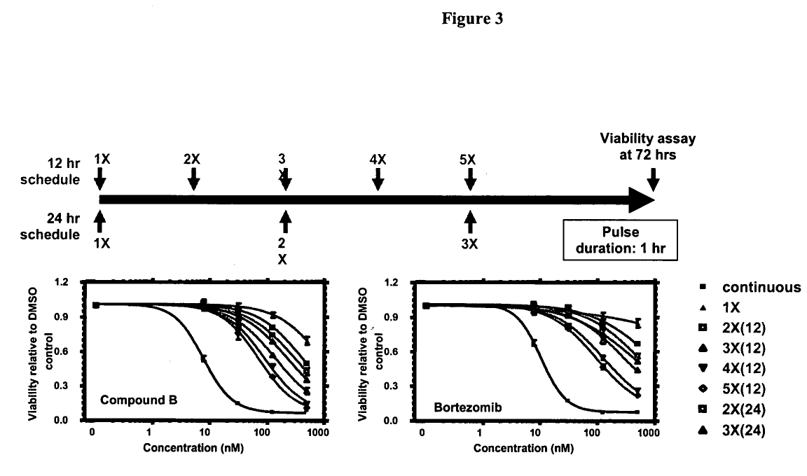

HT29 (a colon adenocarcinoma cell line) cells were treated with either bortezomib or Compound B for 1 hour at various intervals for a 72 hour period, as depicted in FIG. 3. Cell viability was determined at the end of 72 hours.

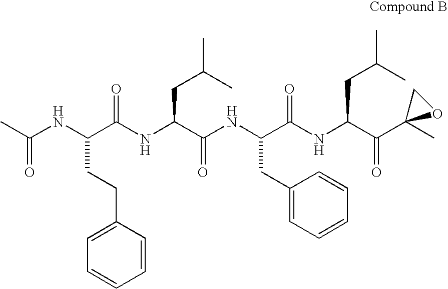

As expected, continuous exposure to either proteasome inhibitor for the entire 72 hour period resulted in the most dramatic cytotoxicity. Both proteasome inhibitors demonstrate an increase in cytotoxicity and decrease in cell viability with increasing number of exposure periods. This result is likely due to an increased amount of time where the proteasome activity is suppressed.

Anti-Tumor Response Against Human Xenografts is Enhanced by Prolonged Proteasome Inhibition.

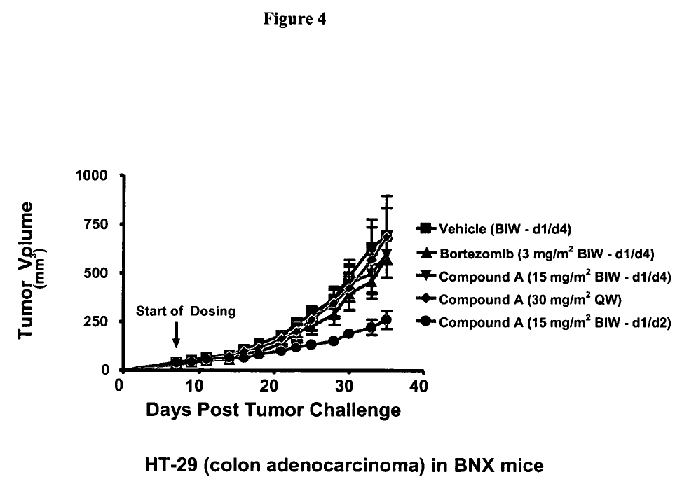

In a human xenograft model using an HT-29 cell line, proteasome inhibitors were administered using the indicated regimens. As shown in FIG. 4, administration of Compound A as a single bolus dose was insufficient to induce an anti-tumor response, as measured by tumor volume. However, the same dose divided into separate doses spread 24 but not 72 h apart resulted in a significant anti-tumor response. Noteworthy, a dose of bortezomib equivalent to the MTD for this strain of mouse, was unable to induce an anti-tumor response when doses were administered on the clinical schedule.

Equivalents

Those skilled in the art will recognize, or be able to ascertain using no more than routine experimentation, numerous equivalents to the compositions and methods of use thereof described herein. Such equivalents are considered to be within the scope of this invention and are covered by the following claims.

All of the above-cited references and publications are hereby incorporated by reference.

What is claimed is:

1. A method for inhibiting proteasome activity in a patient, comprising administering to the patient a dose of a second proteasome inhibitor before proteasome activity in the patient has fully recovered from the previous administration of a dose of a first proteasome inhibitor.

2. The method of claim 1, wherein the first proteasome inhibitor and the second proteasome inhibitor are the same.

3. The method of claim 1, wherein the first proteasome inhibitor and the second proteasome inhibitor are different.

4. The method of claim 3, wherein at least one of the first proteasome inhibitor and the second proteasome inhibitor is bortezomib.

5. The method of claim 1, wherein at least one of the first proteasome inhibitor and the second proteasome inhibitor is a peptide epoxide.

6. The method of claim 3, wherein at least one of the first proteasome inhibitor and the second proteasome inhibitor is lactacystin or an analog thereof.

7. The method of claim 1, wherein the second dose is administered within about 24 hours of the first dose.

8. The method of claim 1, wherein the second dose is administered within about 12 hours of the first dose.

9. The method of claim 1, wherein the second dose is administered when proteasome activity in the patient is 90% or less of the proteasome activity in the patient prior to administration of the first dose.

10. The method of claim 1, further comprising administering a third dose of a proteasome inhibitor before proteasome activity has fully recovered from the administration of the second dose.

11. A method for the treatment of inflammation in a patient, comprising inhibiting proteasome activity in the patient according to the method of claim 1.

12. A method for inhibiting or reducing HIV infection in a patient, comprising inhibiting proteasome activity in the patient according to the method of claim 1.

13. A method for the treatment of neurodegenerative disease in a patient, comprising inhibiting proteasome activity in the patient according to the method of claim 1.

14. A method for the treatment of cancer in a patient, comprising inhibiting proteasome activity in the patient according to the method of claim 1.

15. A method for the treatment of immune related disease in a patient, comprising inhibiting proteasome activity in the patient according to the method of claim 1.

16. A method for affecting the level of viral gene expression in a patient, comprising inhibiting proteasome activity in the patient according to the method of claim 1.

17. A method for altering the variety of antigenic peptides produced by the proteasome in a patient, comprising inhibiting proteasome activity in the patient according to the method of claim 1.

18. A pharmaceutical package, comprising at least one dose of a proteasome inhibitor and instructions for administering a dose of a proteasome inhibitor while proteasome activity in a patient is still at least partly suppressed from prior administration of a dose of a proteasome inhibitor.
